(19) United States
(12) Patent Application Publication
(10) Pub. No.: US 2026/0297052 A1
(43) Pub. Date: Oct. 1, 2026

(54) THIOLACTONES AS MICROCAPSULE PRECURSORS

(71) Applicant: FMC CORPORATION, Philadelphia, PA (US)

(72) Inventors: Sergiy PELESHANKO, Hockessin, DE (US); Christina Stauffer, Landenberg, PA (US); Scott Caplan, Bear, DE (US); Peter Gildner, Newark, DE (US); Kenneth Leavitt, Newark, DE (US); Eric Allen Marshall, Rising Sun, MD (US); Stephen Frederick McCann, Newark, DE (US)

(21) Appl. No.: 19/570,645

(22) Filed: Mar. 18, 2026

Related U.S. Application Data
(60) Provisional application No. 63/774,716, filed on Mar. 19, 2025.

(51) Int. Cl. C07D 333/32; B01J 13/18

(52) U.S. Cl. CPC C07D 333/32; B01J 13/18

(57) ABSTRACT

Disclosed are pre-polymer compounds of Formula 1, including all stereoisomers thereof,

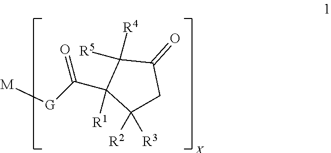

wherein:
X, M, G, R¹, R², R³, R⁴, R⁵ and R⁶ are as defined in the Summary of the Invention.

More particularly, this invention pertains to a pre-polymer compound of Formula 1 including all stereoisomers thereof. This invention also relates to a polymer microcapsule prepared from the compound of Formula 1. This invention includes a formulation comprising the polymer microcapsules consisting of surfactants, solid diluents and liquid diluents. This invention further relates to a process of preparing the microcapsule by interfacial polymerization.

### FIELD OF THE INVENTION

This invention relates to certain microcapsule precursors as pre-polymer compounds, their stereoisomers, and methods of their use for preparing microcapsules.

### BACKGROUND OF THE INVENTION

Encapsulation of agrochemical active ingredients aids in releasing the agrochemical active ingredients in a controlled manner, extending the activity of the agrochemical active ingredients for extended durations, protecting the agrochemical active ingredients from UV radiation, controlling the evaporation of volatile agrochemical active ingredients, and/or avoiding crop injury. Thus, encapsulation of agrochemical active ingredients is beneficial and desirable for numerous reasons.

Polyurea microcapsules produced by interfacial polymerization of isocyanates and amines are widely used to encapsulate agrochemicals. However, polyurea microcapsules do not undergo degradation in the environment. As a result, the widespread use of microplastics like polyurea microcapsules has polluted the marine environment and bans of microplastics are contemplated by regulatory agencies.

Accordingly, there is a need for new microcapsules suitable for use with agrochemicals.

### SUMMARY OF THE INVENTION

This invention is directed to a pre-polymer compound of Formula 1 and stereoisomers thereof, and their use to prepare microcapsules:

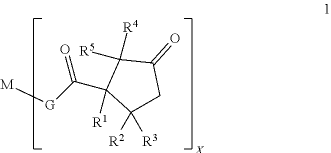

wherein:
X is 2, 3 or 4;
M is a di-, tri- or tetra-valent core moiety;
each G is independently O or NR⁶;
R¹ is hydrogen, C₁-C₄ alkyl or C₃-C₆ cycloalkyl;
R² is hydrogen, C₁-C₄ alkyl or C₃-C₆ cycloalkyl;
R³ is hydrogen, C₁-C₄ alkyl or C₃-C₆ cycloalkyl;
R⁴ is hydrogen, C₁-C₄ alkyl or C₃-C₆ cycloalkyl;
R⁵ is hydrogen, C₁-C₄ alkyl or C₃-C₆ cycloalkyl; and
R⁶ is hydrogen or C₁-C₄ alkyl.

More particularly, this invention pertains to a pre-polymer compound of Formula 1 including all stereoisomers thereof. This invention also relates to a polymer microcapsule prepared from the compound of Formula 1. This invention includes a formulation comprising the polymer microcapsules consisting of surfactants, solid diluents and liquid diluents. This invention further relates to a process of preparing the microcapsule by interfacial polymerization.

### DETAILS OF THE INVENTION

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a compound, composition, mixture, process or method that comprises a list of elements is not necessarily limited to only those elements but may include other elements not expressly listed or inherent to such compound, composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of” Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers. “Cycloalkyl” includes, for example, cyclopropyl, cyclobutyl, cyclopentyl and cyclohexyl.

The total number of carbon atoms in a substituent group is indicated by the “Cᵢ-Cⱼ” prefix where i and j are numbers from 1 to 8. For example, C₁-C₄ alkylsulfonyl designates methylsulfonyl through butylsulfonyl; C₂ alkoxyalkyl designates CH₃OCH₂—; C₃ alkoxyalkyl designates, for example, CH₃CH(OCH₃)—, CH₃OCH₂CH₂— or CH₃CH₂OCH₂—; and C₄ alkoxyalkyl designates the various isomers of an alkyl group substituted with an alkoxy group containing a total of four carbon atoms, examples including CH₃CH₂CH₂OCH₂— and CH₃CH₂OCH₂CH₂—.

When di-, tri- or tetra-valent core moiety comprises a 5- or 6-membered nitrogen-containing heterocyclic ring, it may be attached to the remainder of Formula 1 though any available carbon or nitrogen ring atom, unless otherwise described. As noted above, the di-, tri- or tetra-valent core moiety can be (among others) phenyl optionally substituted with one or more substituents selected from a group of substituents as defined in the Summary of the Invention. An example of phenyl optionally substituted with one to five substituents is the ring illustrated as U-1 in Exhibit 1, wherein R^{v} is R^{A}, R^{B} or R^{C} as defined in the Summary of the Invention for the di-, tri- or tetra-valent core moiety comprises and r is an integer (from 0 to 5).

As noted above, di-, tri- or tetra-valent core moiety comprises can be (among others) a 5- or 6-membered heterocyclic ring, which may be saturated or unsaturated, optionally substituted with one or more substituents selected from a group of substituents as defined in the Summary of the Invention. Examples of a 5- or 6-membered unsaturated aromatic heterocyclic ring optionally substituted with from one or more substituents include the rings U-2 through U-61 illustrated in Exhibit 1 wherein R^{v} is any substituent as defined in the Summary of the Invention for the di-, tri- or tetra-valent core moiety comprises (i.e. R^{A}, R^{B} or R^{C}) and r is an integer from 0 to 4, limited by the number of available positions on each U group. As U-29, U-30, U-36, U-37, U-38, U-39, U-40, U-41, U-42 and U-43 have only one available position, for these U groups r is limited to the integers 0 or 1, and r being 0 means that the U group is unsubstituted and a hydrogen is present at the position indicated by (R^{v})ᵣ.

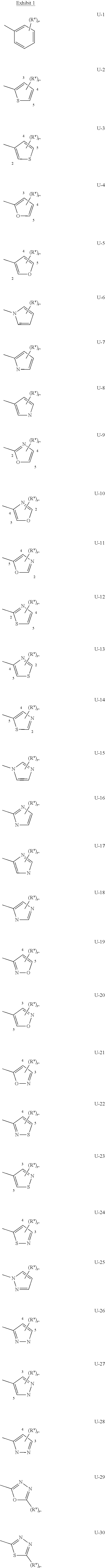

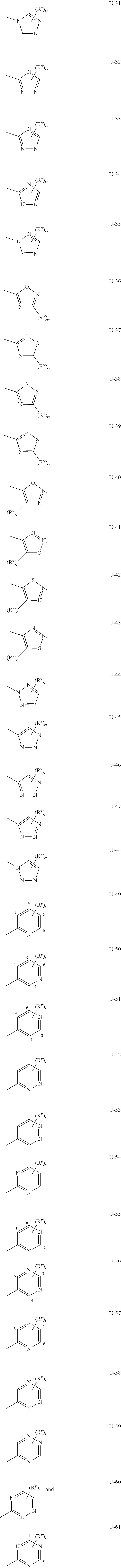

Note that when R^{A}, R^{B} or R^{C} is a 5- or 6-membered saturated or unsaturated non-aromatic heterocyclic ring optionally substituted with one or more substituents selected from the group of substituents as defined in the Summary of the Invention for M, one or two carbon ring members of the heterocycle can optionally be in the oxidized form of a carbonyl moiety.

Examples of a 5- or 6-membered saturated or non-aromatic unsaturated heterocyclic ring containing ring members selected from up to two O atoms and up to two S atoms, and optionally substituted on carbon atom ring members with up to five halogen atoms includes the rings G-1 through G-35 as illustrated in Exhibit 2. Note that when the attachment point on the G group is illustrated as floating, the G group can be attached to the remainder of Formula 1 through any available carbon or nitrogen of the G group by replacement of a hydrogen atom. The optional substituents corresponding to R^{v} can be attached to any available carbon or nitrogen by replacing a hydrogen atom. For these G rings, r is typically an integer from 0 to 4, limited by the number of available positions on each G group.

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this invention can exist as one or more stereoisomers. The various stereoisomers include enantiomers, diastereomers, atropisomers and geometric isomers. Stereoisomers are isomers of identical constitution but differing in the arrangement of their atoms in space and include enantiomers, diastereomers, cis-trans isomers (also known as geometric isomers) and atropisomers. Atropisomers result from restricted rotation about single bonds where the rotational barrier is high enough to permit isolation of the isomeric species. One skilled in the art will appreciate that one stereoisomer may be more active and/or may exhibit beneficial microencapsulation efficiency when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form.

The thiolactone pre-polymer compound of Formula 1 possess a chiral center. The enantiomers are depicted as Formula 1′ and Formula 1″ (where G is defined in the Summary of the Invention) with the chiral center identified with an asterisk (*). For a comprehensive discussion of all aspects of stereoisomerism, see Ernest L. Eliel and Samuel H. Wilen, Stereochemistry of Organic Compounds, John Wiley & Sons, 1994.

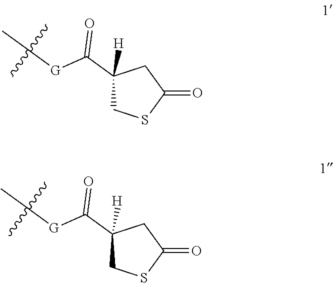

For example, when M is a di-valent core moiety, then pre-polymer compounds of Formula 1 can also possess an additional chiral center. These enantiomers are depicted as Formula 2′ and Formula 2″ with the chiral center identified with an asterisk (*). Various enentiomers can similarly be present when M is tri- or tetra-valent moiety. For a comprehensive discussion of all aspects of stereoisomerism, see Ernest L. Eliel and Samuel H. Wilen, Stereochemistry of Organic Compounds, John Wiley & Sons, 1994.

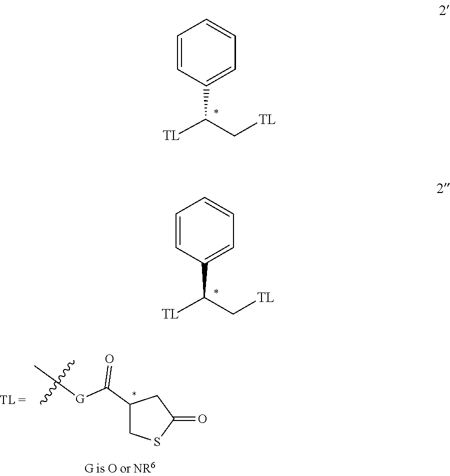

Molecular depictions drawn herein follow standard conventions for depicting stereochemistry. To indicate stereoconfiguration, bonds rising from the plane of the drawing and towards the viewer are denoted by solid wedges wherein the broad end of the wedge is attached to the atom rising from the plane of the drawing towards the viewer. Bonds going below the plane of the drawing and away from the viewer are denoted by dashed wedges wherein the narrow end of the wedge is attached to the atom further away from the viewer. Constant width lines indicate bonds with a direction opposite or neutral relative to bonds shown with solid or dashed wedges; constant width lines also depict bonds in molecules or parts of molecules in which no particular stereoconfiguration is intended to be specified.

Preferably, this invention comprises racemic mixtures, for example, equal amounts of the enantiomers of Formulae 1′ and 1″. In addition, this invention includes compounds that are enriched compared to the racemic mixture in an enantiomer of Formula 1. Also included are the essentially pure enantiomers of compounds of Formula 1, for example, Formula 1′ and Formula 1″.

When enantiomerically enriched, one enantiomer is present in greater amounts than the other, and the extent of enrichment can be defined by an expression of enantiomeric excess (“ee”), which is defined as (2x-1) 100%, where x is the mole fraction of the dominant enantiomer in the mixture (e.g., an ee of 20% corresponds to a 60:40 ratio of enantiomers). The pre-polymer compounds of this invention can have at least a 50% enantiomeric excess; more preferably at least a 75% enantiomeric excess; still more preferably at least a 90% enantiomeric excess; and the most preferably at least a 94% enantiomeric excess of the more active isomer.

Compounds of Formula 1 can comprise additional chiral centers. For example, substituents and other molecular constituents such as R¹, R², R³, R⁴, R⁵ and R⁶ may themselves contain chiral centers. This invention comprises racemic mixtures as well as enriched and essentially pure stereoconfigurations at these additional chiral centers.

Compounds of this invention can exist as one or more conformational isomers due to restricted rotation about any amide bonds present (e.g., in M) in Formula 1. This invention comprises mixtures of conformational isomers. In addition, this invention includes compounds that are enriched in one conformer relative to others.

Compounds of Formula 1 can exist in more than one form, and Formula 1 thus also include all crystalline and non-crystalline forms of the compounds they represent. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound of Formula 1 can exhibit beneficial effects (e.g., suitability for preparation of useful formulations, improved biological performance) relative to another polymorph or a mixture of polymorphs of the same compound of Formula 1. Preparation and isolation of a particular polymorph of a compound of Formula 1 can be achieved by methods known to those skilled in the art including, for example, crystallization using selected solvents and temperatures. For a comprehensive discussion of polymorphism see R. Hilfiker, Ed., Polymorphism in the Pharmaceutical Industry, Wiley-VCH, Weinheim, 2006.

This invention relates to a pre-polymer compound of Formula 1 as defined and described above in the Summary of the Invention. This invention also relates to a method for preparing a microencapsulated formulation from the compound of Formula 1. This invention also relates to a polymerized microencapsulated formulation incorporating an agrochemical compound. This invention is further defined as described in any of the Embodiments below.
Embodiment 1. A pre-polymer compound of Formula 1, stereoisomers thereof, their use as a microcapsule precursor, and their use in the method to prepare microcapsules.
Embodiment 2. The pre-polymer compound of Embodiment 1 wherein X is 2 or 3.
Embodiment 3. The pre-polymer compound of Embodiment 2 wherein X is 2.
Embodiment 4. The pre-polymer compound of Embodiment 2 wherein X is 3.
Embodiment 5. The pre-polymer compound of Embodiment 1 wherein X is 4.
Embodiment 6. The pre-polymer compound of any one of Embodiments 1 through 5 wherein is R¹ is hydrogen or C₁-C₄ alkyl.
Embodiment 7. The pre-polymer compound of Embodiment 6 wherein R¹ is hydrogen, methyl or ethyl.
Embodiment 8. The pre-polymer compound of Embodiment 7 wherein R¹ is hydrogen or methyl.
Embodiment 9. The pre-polymer compound of Embodiment 8 wherein R¹ is methyl.
Embodiment 10. The pre-polymer compound of Embodiment 8 wherein R¹ is hydrogen.
Embodiment 11. The pre-polymer compound of any one of Embodiments 1 through 10 wherein R² is hydrogen, methyl or ethyl.
Embodiment 12. The pre-polymer compound of Embodiment 11 wherein R² is hydrogen or methyl.
Embodiment 13. The pre-polymer compound of Embodiment 12 wherein R² is methyl.
Embodiment 14. The pre-polymer compound of Embodiment 12 wherein R² is hydrogen.
Embodiment 15. The pre-polymer compound of any one of Embodiments 1 through 14 wherein R³ is hydrogen, methyl or ethyl.
Embodiment 16. The pre-polymer compound of Embodiment 15 wherein R³ is hydrogen or methyl.
Embodiment 17. The pre-polymer compound of Embodiment 16 wherein R³ is methyl.
Embodiment 18. The pre-polymer compound of Embodiment 16 wherein R³ is hydrogen.
Embodiment 19. The pre-polymer compound of any one of Embodiments 1 through 18 wherein R⁴ is hydrogen, methyl or ethyl.
Embodiment 20. The pre-polymer compound of Embodiment 19 wherein R⁴ is hydrogen or methyl.
Embodiment 21. The pre-polymer compound of Embodiment 20 wherein R⁴ is methyl.
Embodiment 22. The pre-polymer compound of Embodiment 20 wherein R⁴ is hydrogen.
Embodiment 23. The pre-polymer compound of any one of Embodiments 1 through 22 wherein R⁵ is hydrogen, methyl or ethyl.
Embodiment 24. The pre-polymer compound of Embodiment 23 wherein R⁵ is hydrogen or methyl.
Embodiment 25. The pre-polymer compound of Embodiment 24 wherein R⁵ is methyl.
Embodiment 26. The pre-polymer compound of Embodiment 25 wherein R⁵ is hydrogen.
Embodiment 27. The pre-polymer compound of any one of Embodiments 1 through 26 wherein R⁶ is hydrogen or methyl.
Embodiment 28. The pre-polymer compound of Embodiment 27 wherein R⁶ is hydrogen.
Embodiment 29. The pre-polymer compound of any one of Embodiments 1 through 3, or 6 through 28 wherein M is a di-valent core moiety.
Embodiment 30. The pre-polymer compound of Embodiment 29 wherein M is a di-valent C₂-C₆ alkylene core moiety.
Embodiment 31. The pre-polymer compound of Embodiment 30 wherein M is a di-valent C₂-C₄ alkylene core moiety.
Embodiment 32. The pre-polymer compound of Embodiment 31 wherein M is a di-valent C₂-C₃ alkylene core moiety.
Embodiment 33. The pre-polymer compound of Embodiment 29 wherein the di-valent core moiety comprises phenyl or pyridine, each phenyl or pyridine optionally substituted with up to 5 substituents independently selected from R^{A}; or a 5- to 6-membered fully unsaturated heterocyclic ring or an 6- to 10-membered heteroaromatic bicyclic ring system, each ring or ring system containing ring members selected from carbon atoms and 1 to 4 heteroatoms independently selected from up to 2 O, up to 2 S and up to 4 N atoms, wherein up to 3 carbon ring members are independently selected from C(═O) and C(═S), each ring or ring system optionally substituted with up to 5 substituents independently selected from R^{B} on carbon atom ring members and selected from R^{C} on nitrogen atom ring members; and the two attachment points to G are at any position on the phenyl, pyridine or R^{A}, 5- to 6-membered fully unsaturated heterocyclic ring or 6- to 10-membered heteroaromatic bicyclic ring system or R^{B} or R^{C}.
Embodiment 34. The pre-polymer compound of Embodiment 33 wherein the di-valent core moiety comprises phenyl optionally substituted with up to 5 substituents independently selected from R^{A}; or a 5-membered fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 N atoms, wherein up to 3 carbon ring members are independently selected from C(═O) and C(═S), each ring or ring system optionally substituted with up to 5 substituents independently selected from R^{B} on carbon atom ring members and selected from R^{C} on nitrogen atom ring members; and the two attachment points to G are at any position on the phenyl or R^{A}, 5-membered fully unsaturated heterocyclic ring or 8- to 10-membered heteroaromatic bicyclic ring system or R^{B} or R^{C}.
Embodiment 35. The pre-polymer compound of Embodiment 34 wherein the di-valent core moiety comprises phenyl optionally substituted with up to 5 substituents independently selected from R^{A}; and the two attachment points to G are at any position on the phenyl or R^{A}.
Embodiment 36. The pre-polymer compound of any one of Embodiments 33 through 35 wherein each R^{A}, R^{B} and R^{C} are independendly selected from methyl, ethyl or cyclopropyl.
Embodiment 37. The pre-polymer compound of any one of Embodiments 1, 2, 4, or 5 through 28 wherein M is a tri-valent core moiety.
Embodiment 38. The pre-polymer compound of Embodiment 37 wherein M is a tri-valent C₂-C₈ alkylene core moiety.
Embodiment 39. The pre-polymer compound of Embodiment 38 wherein M is a tri-valent C₂-C₄ alkylene core moiety.
Embodiment 40. The pre-polymer compound of Embodiment 39 wherein M is a tri-valent C₂-C₃ alkylene core moiety.
Embodiment 41. The pre-polymer compound of Embodiment 37 wherein the tri-valent core moiety comprises phenyl or pyridine, each phenyl or pyridine optionally substituted with up to 5 substituents independently selected from R^{A}; or a 5- to 6-membered fully unsaturated heterocyclic ring or an 6- to 10-membered heteroaromatic bicyclic ring system, each ring or ring system containing ring members selected from carbon atoms and 1 to 4 heteroatoms independently selected from up to 2 O, up to 2 S and up to 4 N atoms, wherein up to 3 carbon ring members are independently selected from C(═O) and C(═S), each ring or ring system optionally substituted with up to 5 substituents independently selected from R^{B} on carbon atom ring members and selected from R^{C} on nitrogen atom ring members; and the three attachment points to G are at any position on the phenyl, pyridine, or R^{A}, 5- to 6-membered fully unsaturated heterocyclic ring or 6- to 10-membered heteroaromatic bicyclic ring system or R^{B} or R^{C}.
Embodiment 42. The pre-polymer compound of Embodiment 41 wherein the tri-valent core moiety comprises phenyl optionally substituted with up to 5 substituents independently selected from R^{A}; or a 5-membered fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 N atoms, wherein up to 3 carbon ring members are independently selected from C(═O) and C(═S), each ring or ring system optionally substituted with up to 5 substituents independently selected from R^{B} on carbon atom ring members and selected from R^{C} on nitrogen atom ring members; and the three attachment points to G are at any position on the phenyl or R^{A}, 5-membered fully unsaturated heterocyclic ring or 8- to 10-membered heteroaromatic bicyclic ring system or R^{B} or R^{C}.
Embodiment 43. The pre-polymer compound of Embodiment 42 wherein the tri-valent core moiety comprises phenyl optionally substituted with up to 5 substituents independently selected from R^{A}; and the three attachment points to G are at any position on the phenyl or R^{A}.
Embodiment 44. The pre-polymer compound of any one of Embodiments 37 through 43 wherein each R^{A}, R^{B} and R^{C} are independendly selected from methyl, ethyl or cyclopropyl.
Embodiment 45. The pre-polymer compound of any one of Embodiments 1 or 5 through 28 wherein M is a tetra-valent core moiety.
Embodiment 46. The pre-polymer compound of Embodiment 45 wherein M is a tetra-valent C₂-C₁₀ alkylene core moiety.
Embodiment 47. The pre-polymer compound of Embodiment 46 wherein M is a tetra-valent C₂-C₆ alkylene core moiety.
Embodiment 48. The pre-polymer compound of Embodiment 47 wherein M is a tetra-valent C₂-C₄ alkylene core moiety.
Embodiment 49. The pre-polymer compound of Embodiment 45 wherein the tetra-valent core moiety comprises phenyl or pyridine, each phenyl or pyridine optionally substituted with up to 5 substituents independently selected from R^{A}; or a 5- to 6-membered fully unsaturated heterocyclic ring or an 6- to 10-membered heteroaromatic bicyclic ring system, each ring or ring system containing ring members selected from carbon atoms and 1 to 4 heteroatoms independently selected from up to 2 O, up to 2 S and up to 4 N atoms, wherein up to 3 carbon ring members are independently selected from C(═O) and C(═S), each ring or ring system optionally substituted with up to 5 substituents independently selected from R^{B} on carbon atom ring members and selected from R^{C} on nitrogen atom ring members; and the four attachment points to G are at any position on the phenyl, pyridine, or R^{A}, 5- to 6-membered fully unsaturated heterocyclic ring or 6- to 10-membered heteroaromatic bicyclic ring system or R^{B} or R^{C}.
Embodiment 50. The pre-polymer compound of Embodiment 49 wherein the tetra-valent core moiety comprises phenyl optionally substituted with up to 5 substituents independently selected from R^{A}; or a 5-membered fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 N atoms, wherein up to 3 carbon ring members are independently selected from C(═O) and C(═S), each ring or ring system optionally substituted with up to 5 substituents independently selected from R^{B} on carbon atom ring members and selected from R^{C} on nitrogen atom ring members; and the four attachment points to G are at any position on the phenyl or R^{A}, 5-membered fully unsaturated heterocyclic ring or 8- to 10-membered heteroaromatic bicyclic ring system, or R^{B} or R^{C}.
Embodiment 51. The pre-polymer compound of Embodiment 50 wherein the tetra-valent core moiety comprises phenyl optionally substituted with up to 5 substituents independently selected from R^{A}; and the four attachment points to G are at any position on the phenyl or R^{A}.
Embodiment 52. The pre-polymer compound of any one of Embodiments 45 through 51 wherein each R^{A}, R^{B} and R^{C} are independendly selected from methyl, ethyl or cyclopropyl.
Embodiment 53. The pre-polymer compound of any one of Embodiments 1 through 28 wherein M is selected from the groupcnitn of

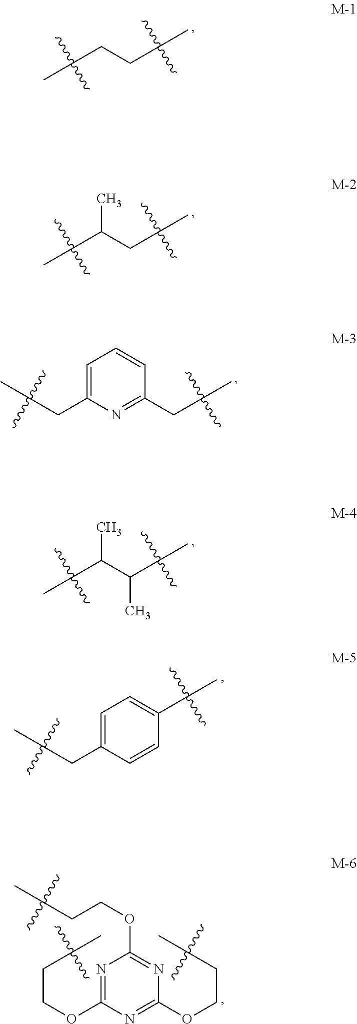

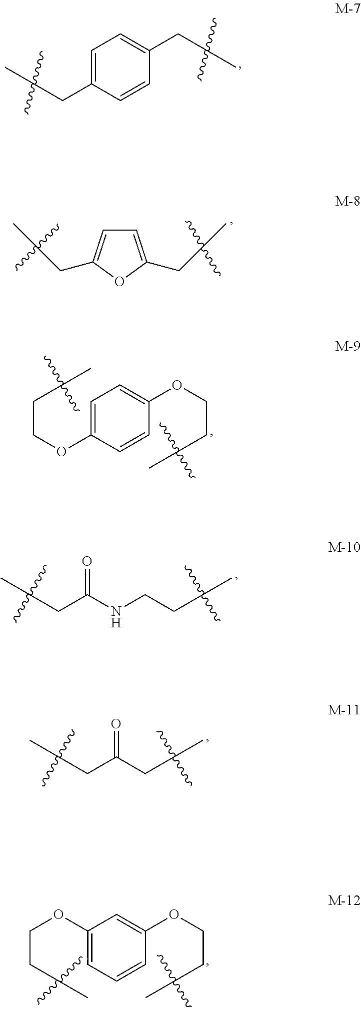 [SMILES: *Cc1ccc(C*)cc1 ; *Cc1ccc(C*)o1 ; *CCOc1ccc(OCC*)cc1 ; *CCNC(=O)C* ; *CC(=O)C* ; *CCOc1cccc(OCC*)c1]

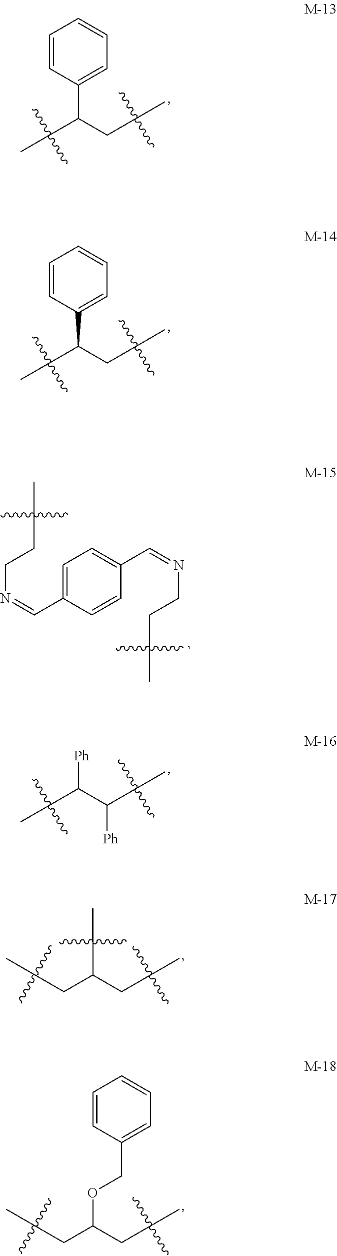 [SMILES: *CC(*)c1ccccc1 ; *C[C@@H](*)c1ccccc1 ; *CC/N=C\c1ccc(/C=N\CC*)cc1 ; *C(c1ccccc1)C(*)c1ccccc1 ; *CC(*)C* ; *CC(C*)OCc1ccccc1]

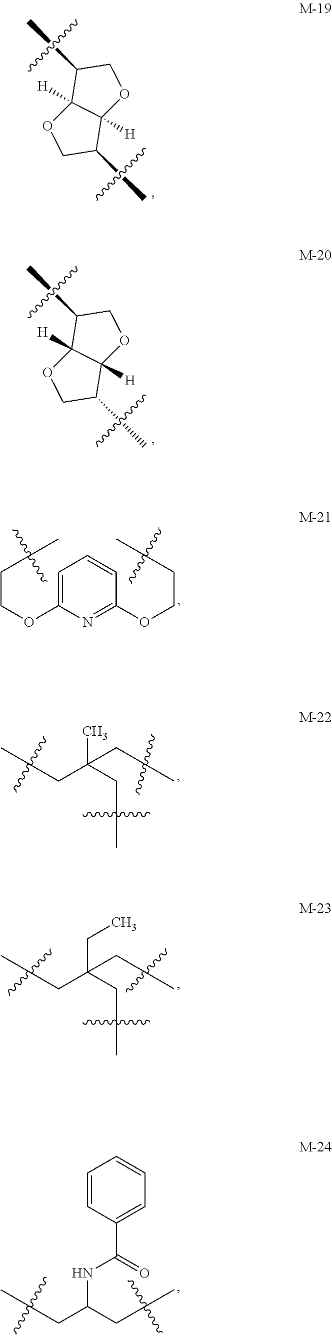

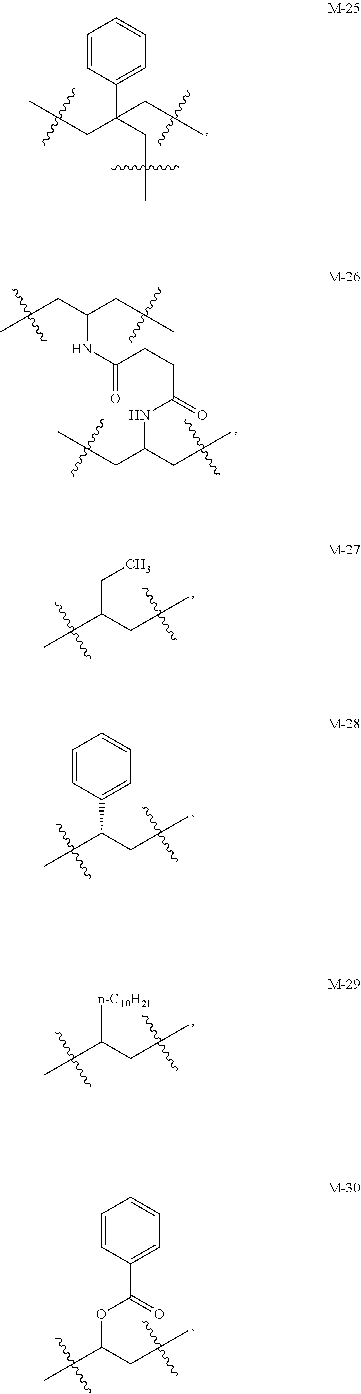

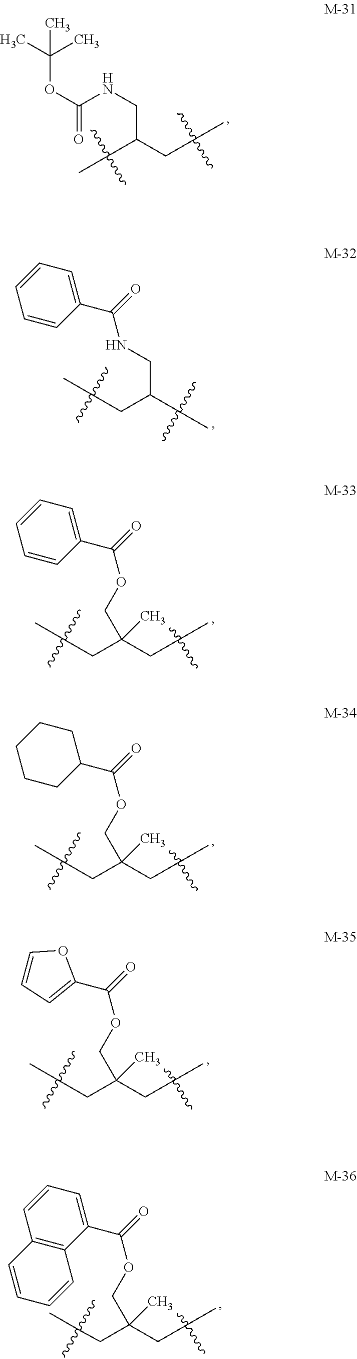 [SMILES: *CC(*)CNC(=O)OC(C)(C)C ; *CC(*)CNC(=O)c1ccccc1 ; *CC(C)(C*)COC(=O)c1ccccc1 ; *CC(C)(C*)COC(=O)C1CCCCC1 ; *CC(C)(C*)COC(=O)c1ccco1 ; *CC(C)(C*)COC(=O)c1cccc2ccccc12]

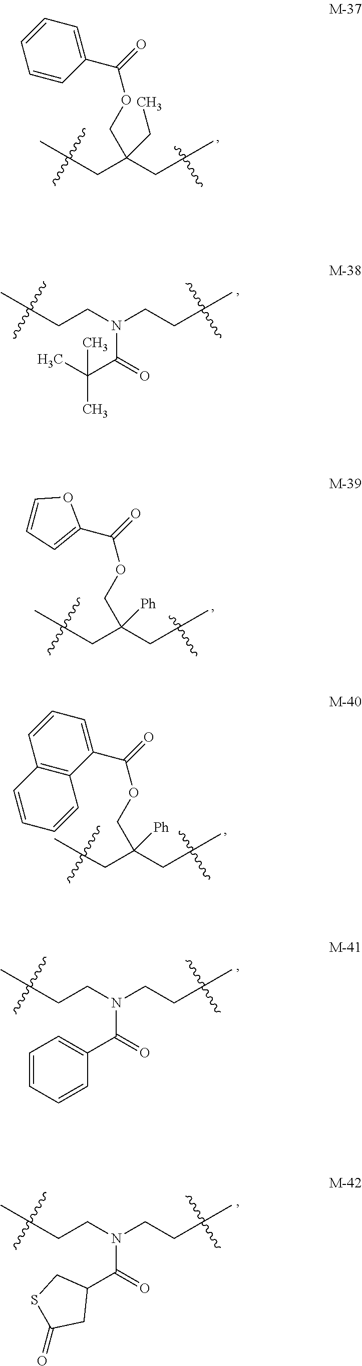 [SMILES: *CC(C*)(CC)COC(=O)c1ccccc1 ; *CCN(CC*)C(=O)C(C)(C)C ; *CC(C*)(COC(=O)c1ccco1)c1ccccc1 ; *CC(C*)(COC(=O)c1cccc2ccccc12)c1ccccc1 ; *CCN(CC*)C(=O)c1ccccc1 ; *CCN(CC*)C(=O)C1CSC(=O)C1]

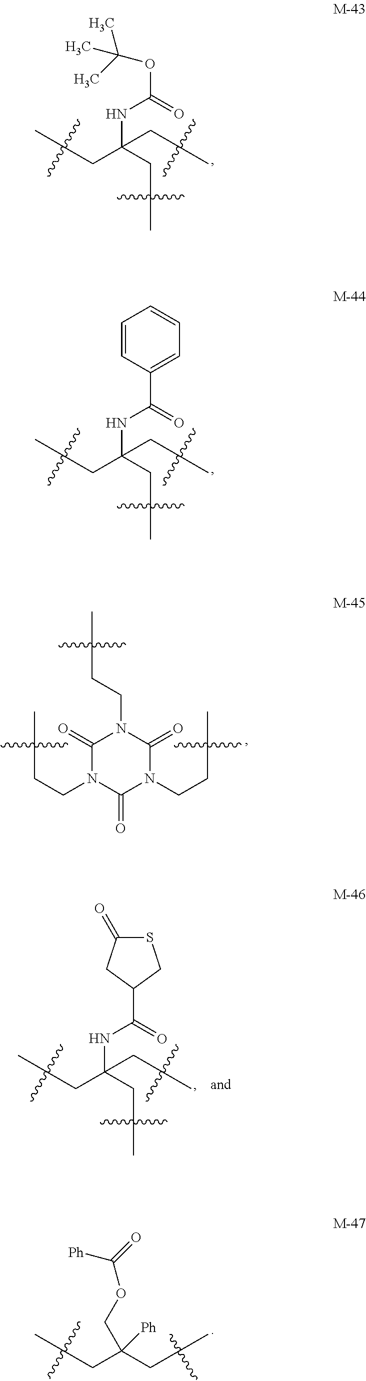 [SMILES: *CC(C*)(C*)NC(=O)OC(C)(C)C ; *CC(C*)(C*)NC(=O)c1ccccc1 ; *CCn1c(=O)n(CC*)c(=O)n(CC*)c1=O ; *CC(C*)(C*)NC(=O)C1CSC(=O)C1 ; *CC(C*)(COC(=O)c1ccccc1)c1ccccc1]

Embodiment 54. A pre-polymer compound of Embodiment 53 wherein M is selected from the group consisting of M-1, M-2, M-3, M-4, M-5, M-7, M-8, M-9, M-10, M-11, M-12, M-13, M-14, M-15, M-16, M-18, M-19, M-20, M-21, M-24, M-27 and M-28.
Embodiment 55. A pre-polymer compound of Embodiment 54 wherein M is selected from the group consisting of M-1, M-2, M-4, M-10, M-11, M-19, M-20 and M-27.
Embodiment 56. A pre-polymer compound of Embodiment 54 wherein M is selected from the group consisting of M-1, M-2, M-4, M-10 and M-11.
Embodiment 57. A pre-polymer compound of Embodiment 54 wherein M is selected from M-3, M-5, M-7, M-8, M-9, M-12, M-13, M-14, M-15, M-16, M-18, M-21, M-24 and M-28.
Embodiment 58. A pre-polymer compound of Embodiment 53 wherein M is selected from the group consisting of M-6, M-17, M-22, M-23 and M-25.
Embodiment 59. A pre-polymer compound of Embodiment 58 wherein M is selected from the group consisting of M-17 and M-23.
Embodiment 60. A pre-polymer compound of Embodiment 58 wherein M is selected from the group consisting of M-6, M-22 and M-25.
Embodiment 61. A pre-polymer compound of Embodiment 53 wherein M is M-26.
Embodiment 62. The pre-polymer compound of Embodiment 53 wherein M is selected from M-1, M-2, M-4, M-13 and M-23.
Embodiment 63. The pre-polymer compound of Embodiment 62 wherein M is selected from M-2, M-13 and M-23.
Embodiment 64. The pre-polymer compound of Embodiment 63 wherein M is M-2.
Embodiment 65. The pre-polymer compound of Embodiment 63 wherein M is M-13.
Embodiment 66. The pre-polymer compound of Embodiment 63 wherein M is M-23.
Embodiment 67. A polymer microcapsule prepared from the pre-polymer compound of Formula 1 as defined in any one of Embodiments 1 through 66 and an amine selected from a di-amine or a tri-amine.
Embodiment 68. The microcapsule described in Embodiment 67 wherein the di-amine or tri-amine comprises an aliphatic di-amine or an aliphatic tri-amine.
Embodiment 69. The microcapsule described in Embodiment 67 wherein the di-amine or tri-amine is selected from polyester di-amines, polyester tri-amines, oligoesteramines, ester diamines, diester diamines, polyester polyols comprising at least two amino groups, and combinations thereof.
Embodiment 70. The microcapsule described in Embodiment 69, wherein the di-amine or tri-amine compound comprises a polyester amine.
Embodiment 71. The microcapsule described in Embodiment 69, wherein the di-amine or tri-amine compound comprises a polyester di-amine or polyester tri-amine.
Embodiment 72. The microcapsule described in Embodiment 69, wherein the di-amine or tri-amine compound comprises an oligoesteramine.
Embodiment 73. The microcapsule described in Embodiment 69, wherein the di-amine or tri-amine compound comprises an ester diamine.
Embodiment 74. The microcapsule described in Embodiment 69, wherein the di-amine or tri-amine compound comprises a diester diamine.
Embodiment 75. The microcapsule described in Embodiment 69, wherein the di-amine or tri-amine compound comprises a polyester polyol comprising at least two amino groups.
Embodiment 76. The polymer microcapsule as defined in Embodiment 67 wherein the amine is a di-amine.
Embodiment 77. The microcapsule of Embodiment 76 wherein the di-amine is selected from the group consisting of ethylenediamine, 1,2-propanediamine, 1,3-propanediamine, 2-methyl-1,2-propanediamine, 2,2-dimethyl-1,3-propanediamine, 1,3-butanediamine, 1,4-butanediamine, 1,3-pentanediamine, 1,5-pentanediamine, 2-butyl-2-ethyl-1,5-pentanediamine, 1,6-hexamethylenediamine (HMDA), methylbis(3-aminopropyl)amine, 1,5-diamino-2-methylpentane (MPMD), 1,2-diaminocyclohexane, 1,3-diaminocyclohexane, 1,4-diaminocyclohexane, bis(4-aminocyclohexyl)methane, bis(4-amino-3-methylcyclohexyl)methane, 1-amino-3-aminomethyl-3,5,5-trimethylcyclohexane (isophoronediamine or IPDA), homologues thereof, isomers thereof, and combinations thereof.
Embodiment 78. The microcapsule of Embodiment 76 wherein the di-amine is selected from the group consisting of ethylenediamine (EDA), 1,3-propanediamine (DAP), 1,6-hexamethylenediamine (HMDA), m-xylylenediamine (XDA), 1,2-diaminocyclohexane (DACH), 2,2-dimethyl-1,3-propanediamine (DMPDA), 1,3-bis(aminomethyl)cyclohexane (BAC), methylene-bis(cyclohexylamine) (MBCHA), 4,4′-methylenebis(2-methylcyclohexylamine) (MBMCHA), and combinations thereof.
Embodiment 79. The microcapsule described in Embodiment 78 wherein the di-amine compound comprises ethylenediamine (EDA).
Embodiment 80. The microcapsule described in Embodiment 78 wherein the di-amine compound comprises 2,2-dimethyl-1,3-propanediamine (DMPDA).
Embodiment 81. The microcapsule described in Embodiment 78 wherein the di-amine compound comprises 1,6-hexamethylenediamine (HMDA).
Embodiment 82. The microcapsule described in Embodiment 78 wherein the di-amine compound comprises m-xylylenediamine (XDA).
Embodiment 83. The microcapsule described in Embodiment 78 wherein the di-amine compound comprises 1,2-diaminocyclohexane (DACH).
Embodiment 84. The microcapsule described in Embodiment 78, wherein the di-amine compound comprises 1,3-bis(aminomethyl)cyclohexane (BAC).
Embodiment 85. The microcapsule described in Embodiment 78, wherein the di-amine compound comprises methylene-bis(cyclohexylamine) (MBCHA).
Embodiment 86. The microcapsule described in Embodiment 77, wherein the di-amine compound comprises 4,4′-methylenebis(2-methylcyclohexylamine) (MBMCHA).
Embodiment 87. The microcapsule described in Embodiment 77, wherein the di-amine compound comprises 1,5-pentanediamine.
Embodiment 88. The microcapsule described in Embodiment 77, wherein the di-amine compound comprises 2-butyl-2-ethyl-1,5-pentanediamine.
Embodiment 89. The microcapsule described in Embodiment 77, wherein the di-amine compound comprises methylbis(3-aminopropyl)amine.
Embodiment 90. The microcapsule described in Embodiment 77, wherein the di-amine compound comprises 1,5-diamino-2-methylpentane (MPMD).
Embodiment 91. The microcapsule described in Embodiment 77, wherein the di-amine compound comprises 1,2-diaminocyclohexane.
Embodiment 92. The microcapsule described in Embodiment 77, wherein the di-amine compound comprises 1,3-diaminocyclohexane.
Embodiment 93. The microcapsule described in Embodiment 77, wherein the di-amine compound comprises 1,4-diaminocyclohexane.
Embodiment 94. The microcapsule described in Embodiment 77, wherein the di-amine compound comprises bis(4-aminocyclohexyl)methane.
Embodiment 95. The microcapsule described in Embodiment 77, wherein the di-amine compound comprises bis(4-amino-3-methylcyclohexyl)methane.
Embodiment 96. The microcapsule described in Embodiment 77, wherein the di-amine compound comprises 1-amino-3-aminomethyl-3,5,5-trimethylcyclohexane (isophoronediamine or IPDA).
Embodiment 97. The polymer microcapsule as defined in Embodiment 67 wherein the amine is a tri-amine.
Embodiment 98. The polymer microcapsule as defined in Embodiment 97 wherein the triamine is 1,3,6 tri-aminohexane.
Embodiment 99. The polymer microcapsule as defined in any one of Embodiments 67 throuth 98 prepared optionally in the presence of a cross-linker.
Embodiment 100. The polymer microcapsule of Embodiment 99 wherein the cross-linker is an acrylate.
Embodiment 101. The polymer microcapsule of Embodiment 99 wherein the cross-linker is a methacrylate.
Embodiment 102. The polymer microcapsule of Embodiment 99 wherein the cross-linker is selected from the group consisting of trimethylolpropane triacrylate (TMPTA), trimethylolpropane trimethacrylate (TMPTMA), ethylene glycol dimethacrylate (EGDA), tricyclo[5.2.1.0-2,6]decanedimethanoldiacrylate (TCDDMDA) and tris[2-(acryloyloxy)ethyl]isocyanurate (TAEI).
Embodiment 103. The polymer microcapsule of Embodiment 99 wherein the cross-linker is selected from the group consisting of trimethylolpropane triacrylate (TMPTA), trimethylolpropane trimethacrylate (TMPTMA), ethylene glycol dimethacrylate (EGDA), tricyclo[5.2.1.0-2,6]decanedimethanoldiacrylate (TCDDMDA).
Embodiment 104. The polymer microcapsule of Embodiment 103 wherein the cross-linker is trimethylolpropane triacrylate (TMPTA).
Embodiment 105. The polymer microcapsule of Embodiment 103 wherein the cross-linker is ethylene glycol dimethacrylate (EGDA).
Embodiment 106. The polymer microcapsule of Embodiment 103 wherein the cross-linker is tricyclo[5.2.1.0-2,6]decanedimethanoldiacrylate (TCDDMDA).
Embodiment 107. The polymer microcapsule as defined in any one of Embodiments 67 through 106 wherein the polymer exhibits a glass transition temperature (T_{g}) of from about 10° C. to about 150° C.
Embodiment 108. The polymer microcapsule as defined in Embodiment 107 wherein the polymer exhibits a glass transition temperature (T_{g}) of from about 20° C. to about 120° C.
Embodiment 109. The polymer microcapsule as defined in Embodiment 108 wherein the polymer exhibits a glass transition temperature (T_{g}) of from about 35° C. to about 100° C.
Embodiment 110. The polymer microcapsule as defined in Embodiment 109 wherein the polymer exhibits a glass transition temperature (T_{g}) of from about 40° C. to about 90° C.
Embodiment 111. A polymer microcapsule prepared from the pre-polymer compound of Formula 1 as defined in any one of Embodiments 67 through 110 wherein the polymer microcapsule exhibits a microencapsulation efficiency of greater than 65%.
Embodiment 112. The polymer microcapsule of Embodiment 111 wherein the polymer microcapsule exhibits a microencapsulation efficiency of greater than 85%.
Embodiment 113. A polymer microcapsule prepared from the pre-polymer compound of Formula 1 as defined in any one of Embodiments 67 through 112 wherein the polymer microcapsule has a particle size D90 of less than 50 microns (micrometers).
Embodiment 114. The polymer microcapsule as defined in Embodiment 113 wherein the polymer microcapsule has a particle size D90 of less than 40 microns.
Embodiment 115. The polymer microcapsule as defined in Embodiment 114 wherein the polymer microcapsule has a particle size D90 of between 5 and 8 microns.
Embodiment 116. The polymer microcapsule as defined in Embodiment 114 wherein the polymer microcapsule encapsulating a pheromone has a particle size D90 of less than 30 microns.
Embodiment 117. The polymer microcapsule as defined in Embodiment 114 wherein the polymer microcapsule encapsulating a herbicide has a particle size D90 of less than 15 microns.
Embodiment 118. The polymer microcapsule as defined in Embodiment 114 wherein the polymer microcapsule encapsulating an insecticide has a particle size D90 of less than 10 microns.
Embodiment 119. A polymer microcapsule prepared from the pre-polymer compound of Formula 1 as defined in any one of Embodiments 1 through 67 wherein the polymer microcapsule encapsulates an agrochemical compound.
Embodiment 120. The polymer microcapsule of Embodiment 119 wherein the agrochemical compound is a herbicide or pheromone.
Embodiment 121. The polymer microcapsule of Embodiment 119 wherein the agrochemical compound is a herbicide.
Embodiment 122. The polymer microcapsule of Embodiment 121 wherein the herbicide is bixlozone, broclozone or clomazone.
Embodiment 123. The polymer microcapsule of Embodiment 120 wherein the agrochemical compound is a pheromone.
Embodiment 123A. The polymer microcapsule of Embodiment 123 wherein the pheromone is (Z)-11-Hexadecenal or (Z)-9-tetradecenyl acetate.
Embodiment 124. The polymer microcapsule of any of Embodiments 67 through 123 wherein the polymer is prepared by a technique selected from the group consisting of solvent evaporation, in-situ polymerization, interfacial polymerization, emulsion polymerization, simple and complex coacervation, layer-by-layer deposition, liposomes, interfacial crosslinking, spray coating, pan coating, and combinations thereof.
Embodiment 125. The polymer microcapsule of Embodiment 124 wherein the polymer is prepared by interfacial polymerization.
Embodiment 126. The polymer microcapsule of Embodiment 120 wherein the polymer is prepared by the process comprising the steps:
   a) forming a mixture comprising:
      at least one thiolactone pre-polymer compound of Formula 1; and
      at least one di-amine or tri-amine compound;
   b) reacting the at least one thiolactone pre-polymer compound of Formula 1 and the at least one di-amine or tri-amine compound to form the polymer reaction product of the at least one thiolactone pre-polymer compound of Formula 1 and the at least one di-amine or tri-amine compound in the presence of an agrochemical compound; and
   c) microencapsulating the agrochemical compound with the reaction product.
Embodiment 127. The polymer microcapsule of Embodiment 121 wherein step b) further comprises a cross-linker.
Embodiment 128. The polymer microcapsule of Embodiment 121 wherein the agrochemical compound is selected from a herbicide, herbicide safener, fungicide, insecticide, nematocide, bactericide, acaricide, growth regulator, pheromone, and plant nutrient.
Embodiment 129. The polymer microcapsule of Embodiment 128 wherein the agrochemical compound is selected from a herbicide, fungicide, insecticide and pheromone.
Embodiment 130. The polymer microcapsule of Embodiment 129 wherein the agrochemical compound is a herbicide.
Embodiment 131. The polymer microcapsule of Embodiment 129 wherein the agrochemical compound is an insecticide.
Embodiment 132. The polymer microcapsule of Embodiment 129 wherein the agrochemical compound is a pheromone.
Embodiment 133. The agrochemical composition described in any of Embodiments 67 through 132 wherein the agrochemical composition is in a form selected from capsule suspension concentrates (CS), mixtures of suspension concentrates (SC) and capsule suspension concentrates (CS) formulations (ZC), flowable concentrates for seed treatment (FS), granules (GR), mixed formulations of capsule suspension concentrates (CS) and aqueous suspo-emulsions (SE) formulations (ZE), mixtures of capsule suspension concentrates (CS) and oil-in-water emulsions (EW) formulations (ZW), wettable powders (WP), and combinations thereof.
Embodiment 134. The agrochemical composition described in Embodiment 133 wherein the agrochemical composition is in the form of a CS formulation or a ZC formulation.
Embodiment 135. The agrochemical composition described in Embodiment 133 wherein the agrochemical composition is in the form of a CS formulation.
Embodiment 136. The agrochemical composition described in Embodiment 133 wherein the agrochemical composition is in the form of a ZC formulation.
Embodiment 137. The agrochemical composition described in Embodiment 133 wherein the agrochemical composition is in the form of a FS formulation.
Embodiment 138. The agrochemical composition described in Embodiment 133 wherein the agrochemical composition is in the form of a GR formulation.
Embodiment 139. The agrochemical composition described in Embodiment 133 wherein the agrochemical composition is in the form of a ZE formulation.
Embodiment 140. The agrochemical composition described in Embodiment 133 wherein the agrochemical composition is in the form of a ZW formulation.
Embodiment 141. The agrochemical composition described in Embodiment 133 wherein the agrochemical composition is in the form of a WP formulation.

Embodiments of this invention, including Embodiments 1-141 above as well as any other embodiments described herein, can be combined in any manner, and the descriptions of variables in the embodiments pertain not only to the pre-polymer compound of Formula 1 but also to the starting compounds and intermediate compounds useful for preparing the pre-polymer compound of Formula 1. In addition, embodiments of this invention, including Embodiments 1-141 above as well as any other embodiments described herein, and any combination thereof, pertain to the polymerized microencapsulated formulation incorporating an agrochemical formulation and methods of using the polymerized microencapsulated formulation incorporating an agrochemical compound.

Combinations of Embodiments 1-141 are illustrated by:
Embodiment PA. The compound of Formula 1 in the Summary of the Invention wherein
   X is 2 or 3;
   M is a di- or tri-valent core moiety;
   R¹ is hydrogen or C₁-C₄ alkyl;
   R² is hydrogen or C₁-C₄ alkyl;
   R³ is hydrogen or C₁-C₄ alkyl;
   R⁴ is hydrogen or C₁-C₄ alkyl;
   R⁵ is hydrogen or C₁-C₄ alkyl;
   R⁶ is hydrogen or methyl.
Embodiment PB. The compound of Embodiment PA wherein
   X is 2;
   M is a di-valent core moiety;
   each G is independently O;
   R¹ is hydrogen or methyl;
   R² is hydrogen or methyl;
   R³ is hydrogen or methyl;
   R⁴ is hydrogen or methyl; and
   R⁵ is hydrogen or methyl.
Embodiment PC. The compound of Embodiment PA wherein
   M is di-valent core moiety comprising phenyl or pyridine, each phenyl or pyridine optionally substituted with up to 5 substituents independently selected from R^{A}; or a 5- to 6-membered fully unsaturated heterocyclic ring or an 6- to 10-membered heteroaromatic bicyclic ring system, each ring or ring system containing ring members selected from carbon atoms and 1 to 4 heteroatoms independently selected from up to 2 O, up to 2 S and up to 4 N atoms, wherein up to 3 carbon ring members are independently selected from C(═O) and C(═S), each ring or ring system optionally substituted with up to 5 substituents independently selected from R^{B} on carbon atom ring members and selected from R^{C} on nitrogen atom ring members; and the two attachment points to G are at any position on the phenyl, pyridine or R^{A}, 5- to 6-membered fully unsaturated heterocyclic ring or 6- to 10-membered heteroaromatic bicyclic ring system or R^{B} or R^{C};
   each R^{A}, R^{B} and R^{C} are independendly selected from methyl, ethyl or cyclopropyl;
   R¹ is hydrogen;
   R² is hydrogen;
   R³ is hydrogen;
   R⁴ is hydrogen; and
   R⁵ is hydrogen.
Embodiment PD. The compound of Embodiment PC wherein
   M is selected from M-1, M-2, M-3, M-4, M-5, M-7, M-8, M-9, M-10, M-11, M-12, M-13, M-14, M-15, M-16, M-18, M-19, M-20, M-21, M-24 and M-27; and each R^{A}, R^{B} and R^{C} are independendly selected from methyl.
Embodiment PE. The compound of Embodiment PA wherein
   X is 3;
   M is a tri-valent core moiety;
   each G is independently O;
   R¹ is hydrogen or methyl;
   R² is hydrogen or methyl;
   R³ is hydrogen or methyl;
   R⁴ is hydrogen or methyl; and
   R⁵ is hydrogen or methyl.
Embodiment PF. The compound of Embodiment PE wherein
   M is selected from the group consisting of M-6, M-17, M-22, M-23 and M-25;
   R¹ is hydrogen;
   R² is hydrogen;
   R³ is hydrogen;
   R⁴ is hydrogen; and
   R⁵ is hydrogen.
Embodiment PG. A polymer microcapsule prepared from the pre-polymer compound of Formula 1 as defined in any one of Embodiments PA through PF and an amine selected from a di-amine or a tri-amine.

Specific Embodiments of a pre-polymer compound of Formula 1 include:
Embodiment PG. A compound of Formula 1 wherein
   X is 2;
   M is selected from the group consisting of M-2 or M-13;
   each G is independently O;
   R¹ is hydrogen;
   R² is hydrogen;
   R³ is hydrogen;
   R⁴ is hydrogen; and
   R⁵ is hydrogen.
Embodiment PG. A compound of Formula 1 wherein
   X is 3;
   M is M-23;
each G is independently O;
   R¹ is hydrogen;
   R² is hydrogen;
   R³ is hydrogen;
   R⁴ is hydrogen; and
   R⁵ is hydrogen.

This invention also relates to a method for controlling undesired vegetation comprising applying to the locus of the vegetation agriculturally effective amounts of the compounds of the invention (e.g., as a composition described herein). Of note as embodiments relating to methods of use are those involving the compounds of embodiments described above. Microencapsules formed with pre-polymers of the invention can be used to microencapsulate agriculturally active compounds for the selective control of pests in crops such as wheat, barley, maize, soybean, sunflower, cotton, oilseed rape and rice, and specialty crops such as sugarcane, citrus, fruit and nut crops.

The compounds of Formula 1 can be prepared by general methods known in the art of synthetic organic chemistry. One or more of the following methods and variations as described in Schemes 1-12 can be used to prepare the compounds of Formula 1. The definitions of X, M, G, R¹, R², R³, R⁴, R⁵ and R⁶ in the compounds of Formulae 1-19 below are as defined above in the Summary of the Invention unless otherwise noted.

Compounds of Formula 1 can be prepared by coupling reactions of carboxylic acid compounds of Formula 2 with compounds of Formula 3 as depicted in Scheme 1.

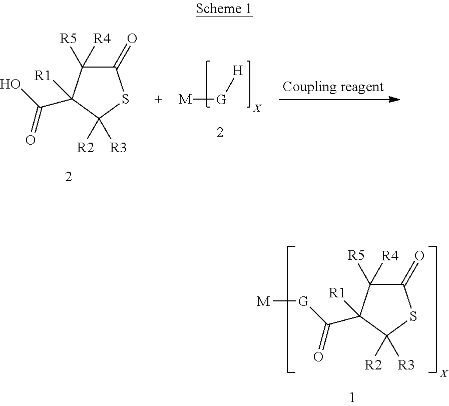

In Scheme 1 reactions, the formation of compounds of Formula 1 when M is a divalent core involve the reaction of two or more molar equivalents of carboxylic acid compounds of Formula 2 with a compound of Formula 3 (where X=2) in the presence of two or more molar equivalents of a coupling reagent such as dicyclohexyl carbodiimide, 1-ethyl-3-(3-dimethylaminopropyl)carbodiimide (EDCI), 1-[bis(dimethylamino)methylene]-1H-1,2,3-triazolo[4,5-b]pyridinium 3-oxid hexafluorophosphate (HATU) or the like. The reaction depicted in Scheme 1 may also involve the use of 0.1 to 5 molar equivalents of a base such as, but not limited to, triethylamine, diisopropylethyamine, or pyridine. A catalyst such as N,N-dimethylaminopyridine or hydroxybenzotriazole can also be used, typically in 0.1 to one molar equivalent. Typical solvents for the reaction depicted in Scheme reactions include, but are not limited to, dichloromethane, tetrahydrofuran, ethyl acetate, N,N-dimethylformamide and toluene at temperatures ranging from −20° C. up to the reflux temperature of the solvent.

In Scheme 1 reactions, when M is a trivalent core (X=3), the use of three or more molar equivalents of the carboxylic acid compound of Formula 2, along with the same coupling reagents previously described can also be employed. In the reactions of Scheme 1, when M is a tetravalent core (X=4), the use of four or more molar equivalents of carboxylic acid compound of Formula 2, along with the same coupling reagents as previously described can also be employed. The bases, catalysts, and solvents for reactions when X=3 or 4 are also the same as previously described.

The compound of Formula 3 (i.e. where X=2) are known in the literature and include diols, diamines and amino alcohols. The compound of Formula 3 where X=3 is known in the literature and include triols, amino diols, diamino alcohols and triamines. The compound of Formula 3 where X=4 is also known in the literature and include tetraols, amino triols, diamino diols, triamino alcohols and tetraamines. When each of R¹, R², R³, R⁴ and R⁵ is hydrogen, the carboxylic acid is 5-oxotetrahydrothiophene-3-carboxylic acid, whose synthesis is described in Tetrahedron, 2016, 72, 6616-6625. The synthesis of a carboxylate of Formula 2 where R¹ is methyl and R², R³, R⁴ and R⁵ are each hydrogen can be found in U.S. Pat. No. 4,803,286.

Alternatively, a compound of Formula 1 can be prepared by the reaction of acid chlorides of Formula 4 with a compound of Formula 3 as depicted in Scheme 2.

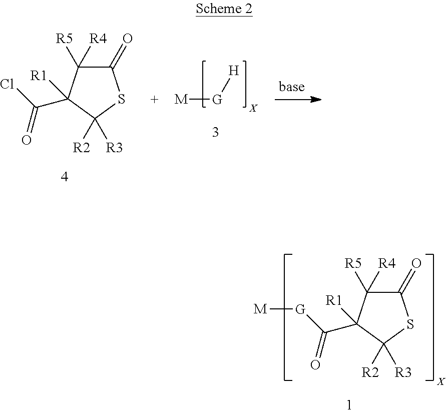

Conditions for the reactions of Scheme 2 are analogous to those described above for the reactions in Scheme 1, except that a coupling reagent is not required. In the reactions depicted in Scheme 2, the formation of a compound of Formula 1 (i.e. when X=2) utilize two or more molar equivalents of the acid chloride compound of Formula 4. The formation of a compound of Formula 1 where X=3 utilize three or more molar equivalents of the acid chloride compound of Formula 4 and the formation of a compound of Formula 1 where X=4 utilize four or more molar equivalents of the acid chloride compound of Formula 4. Suitable bases, catalysts and solvents for Scheme 2 reactions are analogous to those described for Scheme 1 reactions.

The acid chloride compound of Formula 4 can be prepared by the reactions of carboxylic acids of a compound of Formula 2 with reagents such as oxalyl chloride and thionyl chloride using procedures that are known to those skilled in the art.

The preparation of a carboxylic acid compound of Formula 2 can be achieved using procedures illustrated in Scheme 3.

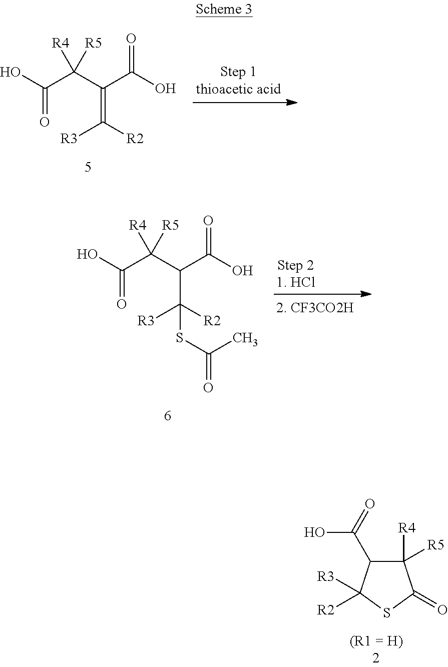

In Step 1 of Scheme 3, thioacetic acid reacts with unsaturated di-acids of a compound of Formula 5 to form the S-acetyl thiol compound of Formula 6. The reaction of Step 1 of Scheme 3 typically involves the use of greater that 1 molar equivalent of thioacetic acid in a solvent such as tetrahydrofuran, ethyl acetate, ethanol, isopropanol or water. An acid scavenger such as triethylamine or pyridine is sometimes used.

In Step 2 of Scheme 3, the S-acetyl thiol compound of Formula 6 reacts with hydrochloric acid or a base and the resulting thiol reacts with trifluoroacetic acid to form the cyclized thiolactone compound of Formula 2. Examples of Scheme 3 reactions can be found in Tetrahedron 2016, 72, 6616-6625 and Bioorg. and Med. Chem. 2009, 17, 1898-1904.

The preparation of unsaturated di-acid compounds of Formula 5 (i.e. where R⁴═R⁵=hydrogen) can be achieved using procedures depicted in Scheme 4.

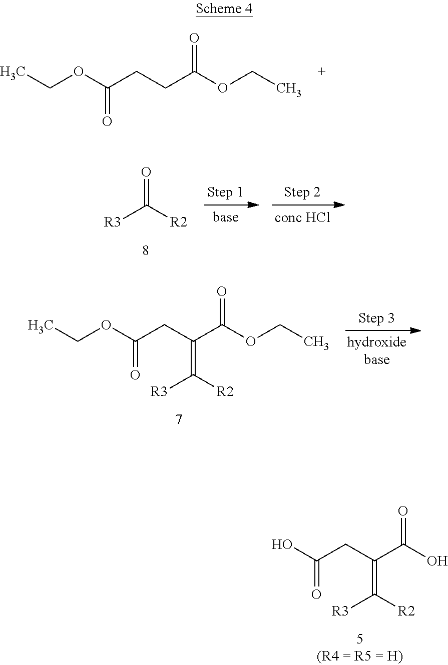

In Step 1 of Scheme 4, diethyl succinate reacts with an aldehyde or ketone of Formula 8 in the presence of a base such as, but not limited to, sodium ethoxide or potassium tert-butoxide to form an intermediate alcohol that is used without purification in Step 2, which involves heating of the intermediate alcohol with alcohol solutions of hydrogen chloride or sulfuric acid. Examples of the formation of the compound of Formula 7 esters may be found in J.O.C 2001, 66, 1914-1918.

In Step 3 of Scheme 4, the diester compound of Formula 7 is hydrolyzed in the presence of a hydroxide base to form the succinic acid compound of Formula 5 using procedures that are well-known to those skilled in the art. For an example, see Bioorg. Med. Chem. 2009, 17, 1898-1904.

The preparation of the unsaturated di-acid compound of Formula 5 where R²=R³=hydrogen can be achieved using procedures depicted in Scheme 5.

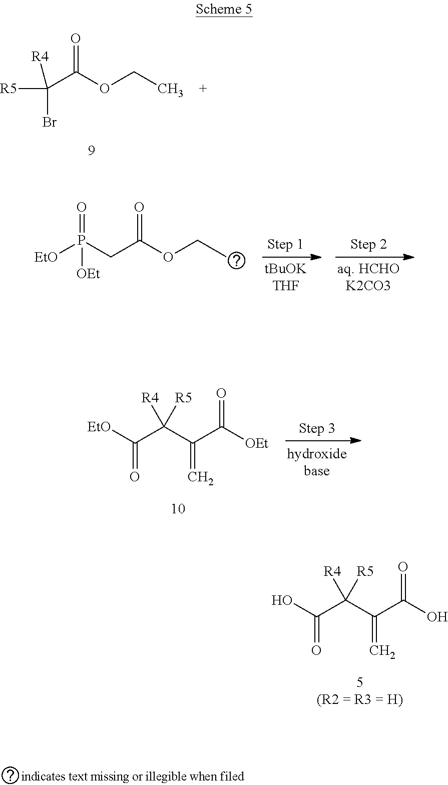

The bromoester compound of Formula 9 reacts with triethyl phosphonoacetate in the presence of potassium tert-butoxide as a solution in tetrahydrofuran to form an intermediate that is not isolated but treated with aqueous formaldehyde and potassium carbonate to form intermediate succinate diester compound of Formula 10. Hydrolysis of the compound of Formula 10 using conditions described in Step 3 of Scheme 4 give the product succinic acid derivative compound of Formula 5 (R²=R³=H). Representative procedures can be found in EP4026562.

Preparation of the compound of Formula 1 (i.e. where X=2) and where the divalent core contains an alternative ester group (i.e. does not contain a thiolactone) can be achieved using procedures outlined in Scheme 6.

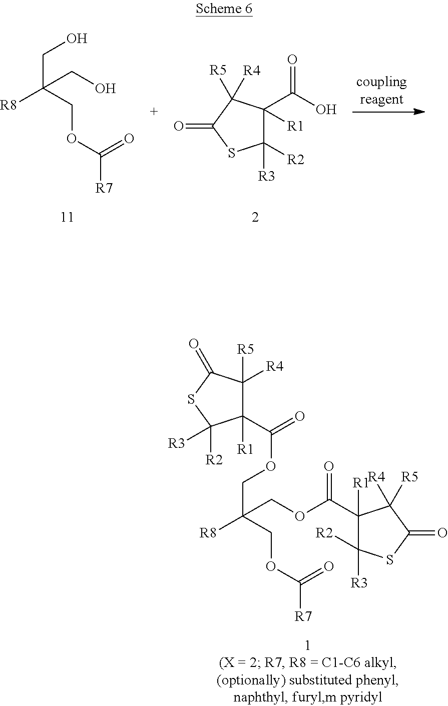

In the reactions depicted in Scheme 6, a triol monoester compound of Formula 11 (where R⁷ and R⁸ are each independently C₁-C₆ alkyl, (optionally) substituted phenyl, naphthyl, furyl or pyridiyl) reacts with greater than 2 molar equivalents of a carboxylic acid compound of Formula 2 in the presence of a coupling reagent. Conditions for the reactions depicted in Scheme 6 are analogous to those previously described for the reactions in Scheme 1. Alternatively, a compound Formula 1 where M is a di-valent core moiety as depicted above in Scheme 6 may be prepared using acid chlorides of Formula 4 using methods analogous to the reactions previously described for Scheme 2.

The preparation of triol monoesters of Formula 11 may be achieved by the hydrolysis of cyclic acetals of Formula 12 as depicted in Scheme 7.

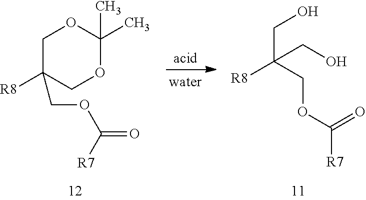

Conditions for acetal hydrolysis are well known to those skilled in the art and include treatment of the acetal with an acid such as hydrochloric acid in a solvent such as water, tetrahydrofuran or acetone. Alternatively, an acidic ion-exchange resin may be used in an alcohol solution. Representative procedures may be found in Carbohydrate Res. Vol 65, 1978, p. 229 and Tetrahedron Lett. Vo. 19, 1978, p. 1623.

The preparations of esters of Formula 12 can be carried out by esterification of an alcohol of Formula 13 with an acid chloride of Formula 14 in the presence of a base as depicted in Scheme 8. Procedures for the esterification of alcohols with acid chlorides are well-known to those skilled in the art.

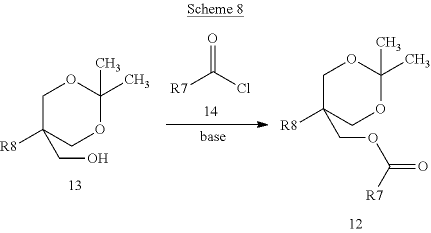

Cyclic acetals of Formula 13 can be prepared by the reactions triols of Formula 15 with 2,2-dimethoxy propane in acetone solution with acid catalysis as depicted in Scheme 9. A representative procedure for Scheme 9 reactions can be found in Chemical Communications 2006, p. 2774.

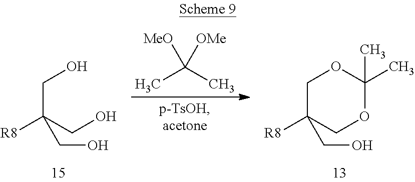

Triols of Formula 15 may often be obtained from commercial sources. Alternatively, they may be prepared using the procedure illustrated in Scheme 10. In Scheme 10 reactions, an aldehyde of Formula 16 reacts with paraformaldehyde in the presence of a base such as calcium hydroxide in a solvent such as tetrahydrofuran. An analogous experimental procedure may be found in Chemische Berichte 1995, 128, 29-34.

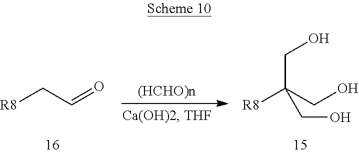

Compounds of Formula 1 with a divalent core that contains an amide substituent may be prepared by methods depicted in Scheme 11.

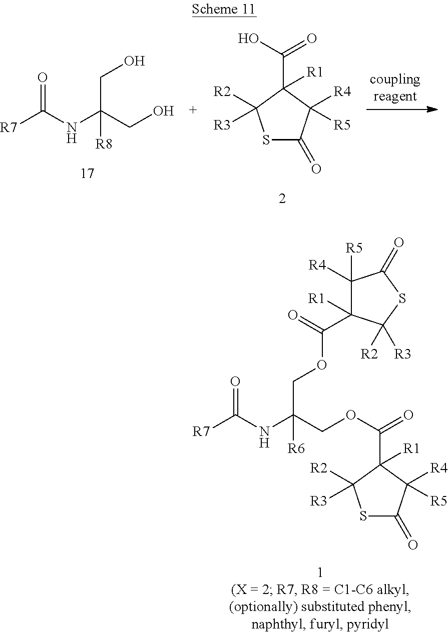

In Scheme 11 reactions, a diol of Formula 17 that contains an amide moiety reacts with two or more molar equivalents of a carboxylic acid 2 in the presence of a coupling reagent. Conditions for Scheme 11 reactions are analogous to those previously described for Scheme 1 reactions.

Compounds of Formula 17 that contain an amide substituent can be prepared by reaction of an amino diol of Formula 18 with an acylation reagent of Formula 19 as depicted in Scheme 12.

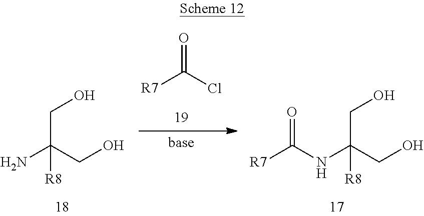

Scheme 12 reactions typically involve the use of one molar equivalent of the acylation reaction of Formula 19 to selectively acylate the amino moiety of 18. Scheme 12 reactions typically involve the use of a base such as, but not limited to, triethylamine, pyridine or potassium carbonate. In addition to acid chlorides of Formula 19, anhydrides analogous to Formula 19 can also be used in Scheme 12 reactions.

It is recognized by one skilled in the art that various functional groups can be converted into others to provide different compounds of Formula 1. For a valuable resource that illustrates the interconversion of functional groups in a simple and straightforward fashion, see Larock, R. C., Comprehensive Organic Transformations: A Guide to Functional Group Preparations, 2nd Ed., Wiley-VCH, New York, 1999. For example, intermediates for the preparation of compounds of Formula 1 may contain aromatic nitro groups, which can be reduced to amino groups, and then be converted via reactions well known in the art such as the Sandmeyer reaction, to various halides, providing compounds of Formula 1. The above reactions can also in many cases be performed in alternate order

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

Polymer microcapsules formed from the pre-polymer compounds of the present invention can be prepared by several methods, including interfacial polymerization. The process involves bringing the pre-polymer into contact with a di-amine or triamine where the pre-polymer is solubilized in an organic (i.e. water-immiscible solvent) layer, and the di-amine or triamine dissolved in an aqueous layer. Polymerization (i.e. microcapsule formation) occurs at the interface between a biphase mixture of said organic (i.e. water-immiscible) and said aqueous layers. The solvent containing the pre-polymer of the present invention comprises at least one water-immiscible solvent (or mixtures thereof), which has a solubility in water of less than 5% by weight at 25° C. Suitable water-immiscible solvents include C8 to C11 aromatic petroleum derivatives (aromatic hydrocarbons) or commercially available solvents under the following brand names: Solvesso® 100, Solvesso® 150, Solvesso® 200, Solvesso® 150ND, Solvesso® 200ND. Additional suitable water-immiscible solvents include vegetable and seed oils selected from liquid triglycerides (e.g., olive oil, kapok oil, castor oil, papaya oil, camellia oil, palm oil, sesame oil, corn oil, rice bran oil, peanut oil, walnut oil, coconut oil, cotton seed oil, soybean oil, rapeseed oil, linseed oil, tung oil, sunflower oil, safflower oil, or their transesterification products thereof, e.g., alkyl esters, such as rapeseed oil methyl ester or rapeseed oil ethyl ester). Suitable water-immiscible solvents include animal-derived oils (e.g., whale oil, cod-liver oil, or mink oil). Suitable, liquid esters of C1-C12 monoalcohols or polyols, for example methanol or ethanol, butanol, n-octanol, i-octanol, dodecanol, cyclopentanol, cyclohexanol, cyclooctanol, ethylene glycol, propylene glycol or benzyl alcohol, with C6-C10 carboxylic or polycarboxylic acids, such as caproic acid, capric acid, caprylic acid and pelargonic acid; or with aromatic carboxylic acids such as benzoic acid, toluic acid, salicylic acid and phthalic acid; hydrocarbons such as aromatic depleted, linear paraffinic, isoparaffinic, cycloparaffinic having a flash point between 40° C. and 250° C. and a distillation range between 150° C. and 450° C., esters, such as terpenoid esters (for example isobornyl acetate), benzyl acetate, benzyl benzoate, butyl benzoate; phosphate ester such as tributyl phosphate, trioctyl phosphate; and liquid amides of C₁-C₃ amines, alkylamines or alkanolamines with C6-C18 carboxylic acids; or mixtures thereof such as N,N-dialkyl alkylamides, preferably fatty acid dimethylamides, more preferably N,N-dimethyl octanamide and/or N,N-dimethyl decanamide and/or N,N-dimethyl dodecanamide and n-octyl-2-pyrrolidone (NOP), N-decyl-2-pyrrolidone (NDP), N-dodecyl-2-pyrrolidone (NDDP), and the morpholine C6-12 Acyl amide derivatives (i.e. the morpholine C8-C10 fatty acids such as JEFFSOL® AG-1730 from Huntsman). Preferred solvents are selected from Solvesso® 200ND), benzyl acetate, N,N-dimethyl dodecanamide, and JEFFSOL® AG-1730.

The interfacial polymerization is optionally performed in the presence of a cross-linker which can be selected from multifunctional acrylates. Multifunctional (meth)acrylate crosslinkers are defined to be multifunctional or methacrylate monomers and include, by way of illustration and not limitation, mono-; di-; tri- or tetra-functional acrylate esters and methacrylate esters. Useful multifunctional (meth)acrylate monomers in the invention are one or more di- and poly-functional acrylate esters, difunctional (meth)acrylate esters, polyfunctional (meth)acrylate esters, and used alone or in combination as blends. In one aspect of the invention multi-functional acrylates or methacrylates can include, by way of illustration, ethylene glycol dimethacrylate, ethylene glycol diacrylate, 1,3-butylene glycol diacrylate; 1,3-butylene glycol dimethacrylate; neopentyl glycol diacrylate; 1,4-butanediol dimethacrylate; 1,4-butaneidiol diacrylate; 1,6-hexanediol diacrylate; 1,6-hexanediol dimethacrylate; trimethylolpropane trimethacrylate; trimethylolpropane triacrylate, tricyclo[5.2.1.0-2,6]decanedimethanoldiacrylate (TCDDMDA) and tris[2-(acryloyloxy)ethyl]isocyanurate (TAEI), or blends of any of the foregoing. Preferred acrylates are selected from the group consisting of neopentyl glycol diacrylate, 1,4-butaneidiol diacrylate, 1,6-hexanediol diacrylate, trimethylolpropane triacrylate and tris[2-(acryloyloxy)ethyl]isocyanurate (TAEI). Most preferred is trimethylolpropane triacrylate.

Without further elaboration, it is believed that one skilled in the art using the preceding description can utilize the present invention to its fullest extent. The following non-limiting Examples are illustrative of the invention. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; “d” means doublet, “t” means triplet, “m” means multiplet, and “dd” means doublet of doublets. Mass spectra (MS) are reported as the molecular weight of the highest isotopic abundance parent ion (M+1) formed by addition of H+(molecular weight of 1) to the molecule, or (M-1) formed by the loss of H+(molecular weight of 1) from the molecule, observed by using liquid chromatography coupled to a mass spectrometer (LCMS) using atmospheric pressure chemical ionization (AP+).

### SYNTHESIS EXAMPLE 1

### Preparation of Compound 22

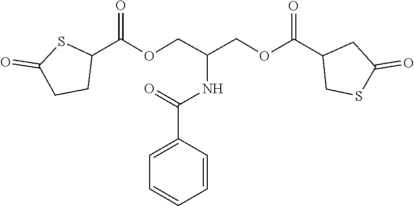 [SMILES: O=C1CC(C(=O)OCC(COC(=O)C2CCC(=O)S2)NC(=O)c2ccccc2)CS1]

To a mixture of N-(1,3-dihydroxypropan-2-yl)benzamide (1.62 g, 8.30 mmol, 1 eq.) and 5-oxothiolane-3-carboxylic acid (2.547 g, 17.427 mmol, 2.1 eq.) under nitrogen atmosphere was added dichloromethane (24.3 mL, 0.341 M) and N,N-dimethylformamide (24.3 mL, 0.341 M) giving a homogenous pink solution. 4-(Dimethylamino)pyridine (0.203 g, 1.66 mmol, 0.2 eq.) and ethyl dimethylaminopropyl carbodiimide (3.341 g, 17.427 mmol, 2.1 eq.) together as solids and stirred the resulting homogenous pale-yellow solution for 18 h to yield a dark purple homogenous solution. Excess solvent was removed under reduced pressure and the residue was partitioned between ethyl acetate and saturated aqueous ammonium chloride solution. The organic layer was separated and washed with saturated aqueous sodium bicarbonate, brine, then dried over anhydrous magnesium sulfate and the solvent was removed under reduced pressure to provide the title compound as a thick purple oil which was purified by silica gel chromatography eluting with ethyl acetate. The resulting yellow pale-yellow oil was dried in a vaccum oven for 18 h (3.49 g, 93.15%).

¹H NMR (500 MHz, DMSO-d₆) δ 2.82 (m, 4H), 3.63 (m, 6H), 4.28 (m, 4H), 4.57 (m, 1H), 7.49 (m, 2H), 7.55 (m, 1H), 7.82 (m, 2H), 8.49 (m, 1H).

### SYNTHESIS EXAMPLE 2

### Preparation of Compound 24

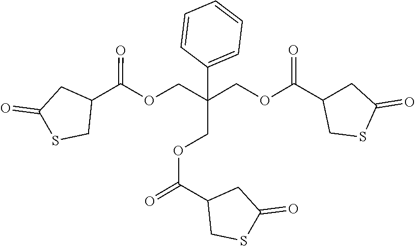 [SMILES: O=C1CC(C(=O)OCC(COC(=O)C2CSC(=O)C2)(COC(=O)C2CSC(=O)C2)c2ccccc2)CS1]

To a mixture of 2-(hydroxymethyl)-2-phenylpropane-1,3-diol (1.0 g, 5.5 mmol, prepared as described in Chemische Berichte, 1995, 128, #1, 29-34), 5-oxothiolane-3-carboxylic acid (2.5 g, 17.0 mmol, prepared as described in Tetrahedron, 2016, 72, #42, 6616-6625), and dichloromethane (55 mL) was added 1-ethyl-3-(3-dimethylaminopropyl)carbodiimide (EDCI) (4.2 g, 22.0 mmol) and 4-dimethyaminopyridine (0.07 g, 0.9 mmol) sequentially at 0° C. The resulting solution was stirred at 25° C. for 16 h, at which time the reaction mixture was partitioned between dichloromethane (100 mL) and saturated aqueous ammonium chloride solution (100 mL). The resulting organic layer was washed with a mixture of saturated aqueous sodium bicarbonate and water (1:3, v:v, 100 mL) and dried over anhydrous magnesium sulfate. After filtration, the resulting solution was treated with 14 g of Celite (diatomaceous earth filter aid) and the resulting mixture was concentrated under reduced pressure. The residue was purified by chromatography on an 80 g silica column eluting with a gradient of 0% to 100% ethyl acetate in hexanes to provide the subject compound as a pale yellow, viscous oil (1.1 g, 35.4% yield).

¹H NMR (500 MHz, CDCl3) δ 7.41 (distorted t, 2H), 7.34 (distorted t, 1H), 7.28 (distorted d, 2H), 4.57-4.50 (m, 6H), 3.51-3.42 (m, 6H), 3.37-3.31 (m, 3H), 2.81-2.73 (m, 3H), 2.67 (distorted dd, 3H).

### SYNTHESIS EXAMPLE 3

### Preparation of Compound 25

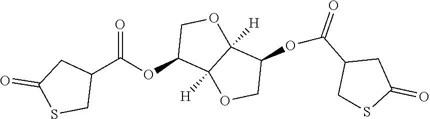 [SMILES: O=C1CC(C(=O)O[C@H]2CO[C@@H]3[C@H]2OC[C@@H]3OC(=O)C2CSC(=O)C2)CS1]

To a stirred solution of isomannide (500 mg, 3.421 mmol, 1 eq.) and 5-oxothiolane-3-carboxylic acid (1.05 g, 7.185 mmol, 2.1 eq.) in anhydrous dichloromethane (30 mL, 0.114 M, 60 Vols) at 0° C. was added 4-(N,N-dimethyl amino)pyri dine (0.021 g, 0.171 mmol, 0.05 eq.) and 1-ethyl-3-(3-dimethylaminopropyl)carbodiimide (EDCI) (1.508 g, 7.869 mmol, 2.3 eq.).

The mixture was allowed to warm to ambient temperature and stirred for 22 h. The mixture was diluted with dichloromethane (30 mL) and washed with aqueous 1 N hydrochloric acid (2×20 mL). The organic layer was washed with saturated aqueous sodium bicarbonate (2×20 mL). The organic layer was dried over magnesium sulfate, filtered, and concentrated under reduced pressure. The resultant oily solid was dried in a vacuum oven at 50° C. for 2 h then for 72 h at ambient temperature to afford the subject compound (1.370 g, 3.404 mmol, 99% yield, 95% NMR purity) as a grey solid. (M. S.=403.3).

### SYNTHESIS EXAMPLE 4

### Preparation of Compound 36

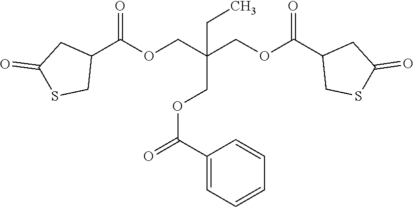 [SMILES: CCC(COC(=O)c1ccccc1)(COC(=O)C1CSC(=O)C1)COC(=O)C1CSC(=O)C1]

A 500 mL round bottom flask was charged with 2-(benzyloxy)propane-1,3-diol (10 g, 54.879 mmol, 1 eq.), 5-oxothiolane-3-carboxylic acid (17.646 g, 120.734 mmol, 2.2 eq.) and dichloromethane (365.86 mL, 0.15 M, 36.586 Vols) and cooled to −10° C. 1-Ethyl-3-(3-dimethylaminopropyl)carbodiimide (EDCI) (26.301 g, 137.198 mmol, 2.5 eq.) and 4-(N,N-dimethylamino)pyridine (1.341 g, 10.976 mmol, 0.2 eq.) were added and the reaction was allowed to warm to room temperature. The reaction was allowed to stir at ambient temperature for 16 h. Aqueous NH₄Cl solution was added, and the reaction was stirred for 5 min. The contents were poured onto a celite extraction tube and eluted with dichloromethane. The crude material was purified by chromatography eluting with ethyl acetate in hexanes to provide 1.0 g of an oil, a compound of the invention.

### SYNTHESIS EXAMPLE 5

### Preparation of Compound 3

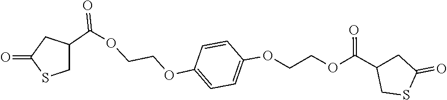 [SMILES: O=C1CC(C(=O)OCCOc2ccc(OCCOC(=O)C3CSC(=O)C3)cc2)CS1]

To a stirred solution of 2-[4-(2-hydroxyethoxy)phenoxy]ethanol (500 mg, 2.522 mmol, 1 eq.) and 5-oxothiolane-3-carboxylic acid (0.774 g, 5.297 mmol, 2.1 eq.) in anhydrous dichloromethane (24 mL, 0.105 M, 48 Vols) at 0° C. was added 4-(N,N-dimethylamino)pyridine (0.015 g, 0.126 mmol, 0.05 eq.) and EDCI (1.209 g, 6.306 mmol, 2.5 eq.). The mixture was allowed to warm to ambient temperature and stirred for 18 h. The mixture was filtered and the collected solid was rinsed with dichloromethane (30 mL). The solid was dried in a vacuum oven at 50° C. for 2 h, then at ambient temperature for 72 h to afford the subject compound (0.932 g, 1.985 mmol, 79% yield, 96.8 wt % purity) as a white solid. (M.S.=455.4)

### SYNTHESIS EXAMPLE 6

### Preparation of Compound 6

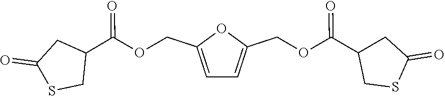 [SMILES: O=C1CC(C(=O)OCc2ccc(COC(=O)C3CSC(=O)C3)o2)CS1]

To a stirred solution of 2,5-bis(hydroxymethyl)furan (9.00 g, 70.243 mmol, 1 eq.) and 5-oxothiolane-3-carboxylic acid (21.560 g, 147.51 mmol, 2.1 eq.) in anhydrous dichloromethane (600 mL, 0.117 M, 66.667 Vols) at 0° C. was added 4-(N,N-dimethylamino)pyridine (0.430 g, 3.52 mmol, 0.05 eq.) and EDCI (31 g, 161.7 mmol, 2.3 eq.). The mixture was allowed to warm to ambient temperature and stirred for 20 h. The mixture was washed with aqueous 1 N HCl (3×100 mL). The organic layer was then washed with saturated aqueous sodium bicarbonate (100 mL). The organic layer was dried over magnesium sulfate, concentrated under reduced pressure, and dried under vacuum overnight to afford the subject compound (24.85 g, 62.639 mmol, 89% yield, 96.9 wt % purity) as a white solid. (M.S.=385.2)

### SYNTHESIS EXAMPLE 7

### Preparation of Compound 7

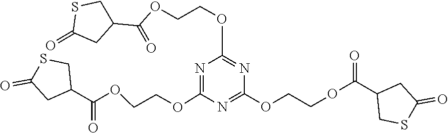 [SMILES: O=C1CC(C(=O)OCCOc2nc(OCCOC(=O)C3CSC(=O)C3)nc(OCCOC(=O)C3CSC(=O)C3)n2)CS1]

To a stirred solution of 2,2′,2″-[1,3,5-triazine-2,4,6-triyltris(oxy)]tris[ethanol](500 mg, 1.914 mmol, 1 eq.) and 5-oxothiolane-3-carboxylic acid (896 mg, 6.13 mmol, 3.203 eq.) in anhydrous dichloromethane (20 mL, 0.096 M, 40 Vols) at 0° C. was added 4-(N,N-dimethylamino)pyridine (0.012 g, 0.098 mmol, 0.05 eq.) and EDCI (1.28 g, 6.677 mmol, 3.5 eq.). The mixture was allowed to warm to ambient temperature and stirred for 18 h. The mixture was diluted with dichloromethane (30 mL) and washed with aqueous 1 N HCl (2×20 mL). The organic layer was washed with saturated aqueous sodium bicarbonate (20 mL). The organic layer was dried over magnesium sulfate and concentrated under reduced pressure to afford a clear, colorless oil. Upon standing the oil began to forma white solid which was triturated with methyl tert-butyl ether (20 mL) for 1 h. The suspension was filtered and rinsed with MTBE (20 mL). The collected solid was dried in vacuum oven at 50° C. for 18 h to afford the subject compound (1.19 g, 1.751 mmol, 91% yield, 95% NMR purity) as a white solid. (MS=646.5)

### SYNTHESIS EXAMPLE 8

### Preparation of Compound 23

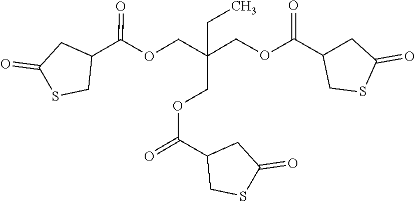 [SMILES: CCC(COC(=O)C1CSC(=O)C1)(COC(=O)C1CSC(=O)C1)COC(=O)C1CSC(=O)C1]

To a mixture of 5-oxothiolane-3-carboxylic acid (29.71 g, 201.2 mmol, 3 eq.), trimethylolpropane (9 g, 67 mmol, 1 eq), and 4-(dimethylamino)pyridine (0.819 g, 6.708 mmol, 0.1 eq.) under nitrogen atmosphere was added ethyl acetate (650 mL, 0.103 M). The reaction was cooled on an ice-water bath then dicyclohexylcarbodiimide (45.67 g, 221.4 mmol, 3.3 eq.) was added as a solid. The reaction was warmed to ambient temperature and stirred for 24 h. The suspended solids were removed by filtration and the liquid concentrated under reduced pressure. The crude product was purified on a silica gel column chromatography eluting with ethyl acetate in hexanes to give the title compound as a white solid (29.7 g, 72.6%). M.S. (M+H)=519.1

### SYNTHESIS EXAMPLE 9

### Preparation of Compound 15

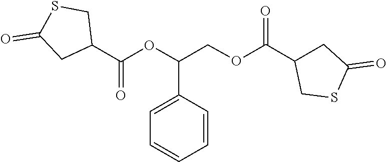 [SMILES: O=C1CC(C(=O)OCC(OC(=O)C2CSC(=O)C2)c2ccccc2)CS1]

To a mixture of 5-oxothiolane-3-carboxylic acid (25.65 g, 173.7 mmol, 2 eq), 1-phenylethane-1,2-diol (12 g, 86.9 mmol, 1 eq), and 4-(dimethylamino)pyridine (0.531 g, 4.343 mmol, 0.05 eq) under nitrogen atmosphere was added ethyl acetate (580 mL, 0.15 M). The reaction was cooled in an ice-water bath then dicyclohexylcarbodiimide (39.42 g, 191.1 mmol, 2.2 eq) was added as a solid. The reaction was warmed to ambient temperature and stirred for 24 h. The suspended solids were removed by filtration and the filtrate liquid was concentrated under reduced pressure. The crude product was purified by silica gel column chromatography eluting with ethyl acetate in hexanes to give the title compound as a viscous liquid (29.0 g, 82.9%). MS (M+H)=395.1

### SYNTHESIS EXAMPLE 10

### Preparation of Compound 12

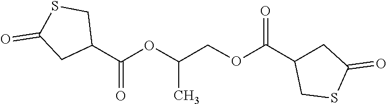 [SMILES: CC(COC(=O)C1CSC(=O)C1)OC(=O)C1CSC(=O)C1]

A solution of 14.79 g (101.89 mmol) of 5-oxothiolane-3-carboxylic acid in 306 mL of dichloromethane was added cooled to −10° C. and 22.04 g (114.99 mmol) of 1-ethyl-3-(3-dimethylaminopropyl)carbodiimide (EDCI) was added. The solution was stirred for 15 min, then 3.5 g (46.00 mmol) of 1,2-propanediol dissolved in 20 mL of dichloromethane was added dropwise over 5 m. 1.124 g (9.2 mmol) of 4-(N,N-dimethylamino)pyridine were added and stirring is continued for 30 min at −10° C. The ice bath is removed, and the reaction is allowed to warm to room temperature and stirred for 1 h. 100 mL of saturated ammonium chloride is added, and the reaction is stirred for 5 min. The organic phase is separated and washed with additional ammonium chloride. The organic phase is collected, dried over magnesium sulfate, filtered, and concentrated to provide 15.4 g of crude product. The crude material was purified by silica gel chromoatography eluting with a gradient of ethl acetate in hexanes to provide 13.99 g of the title compound.

¹H NMR (CHLOROFORM-d) δ 5.19-5.31 (m, 1H), 4.30-4.43 (m, 1H), 4.06-4.17 (m, 1H), 3.54-3.70 (m, 4H), 3.35-3.46 (m, 2H), 2.87-2.98 (m, 2H), 2.71-2.82 (m, 2H), 1.30 (d, 3H).

The microencapsules of this invention will generally be used in an agricultural composition, i.e. formulation, with at least one additional component selected from the group consisting of surfactants, solid diluents and liquid diluents, which serves as a carrier. The formulation or composition ingredients are selected to be consistent with the physical properties of the active ingredient, mode of application and environmental factors such as soil type, moisture and temperature.

The general types of solid compositions are dusts, powders, granules, pellets, prills, pastilles, tablets, filled films (including seed coatings) and the like, which can be water-dispersible (“wettable”) or water-soluble. Active ingredient can be microencapsulated and further formed into a suspension or solid formulation; alternatively the entire formulation of active ingredient can be microencapsulated (or “overcoated”). Microencapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water, but occasionally another suitable medium like an aromatic or paraffinic hydrocarbon or vegetable oil. Spray volumes can range from about from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting.

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, New Jersey.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), alkyl phosphates (e.g., triethyl phosphate), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters, alkyl and aryl benzoates and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol, cresol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof, amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as NN-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures ofnonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

In one common embodiment, microencapsules of the invention are applied, typically in a formulated composition, to a locus comprising desired vegetation (e.g., crops) and undesired vegetation (i.e. weeds), both of which may be seeds, seedlings and/or larger plants, in contact with a growth medium (e.g., soil). In this locus, a composition comprising a compound of the invention can be directly applied to a plant or a part thereof, particularly of the undesired vegetation, and/or to the growth medium in contact with the plant.

Plant varieties and cultivars of the desired vegetation in the locus treated with a compound of the invention can be obtained by conventional propagation and breeding methods or by genetic engineering methods. Genetically modified plants (transgenic plants) are those in which a heterologous gene (transgene) has been stably integrated into the plant's genome. A transgene that is defined by its particular location in the plant genome is called a transformation or transgenic event.

A pre-polymer of this invention can also be used to microencapsulate other biologically active compounds or agents including herbicides, herbicide safeners, fungicides, insecticides, nematocides, bactericides, acaricides, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection.

Examples of herbicide active ingredients are allidochlor, acetochlor, acifluorfen and its sodium salt, aclonifen, acrolein (2-propenal), alachlor, alloxydim, ametryn, amicarbazone, amidosulfuron, aminocyclopyrachlor and its methyl and ethyl esters and its sodium and potassium salts, aminopyralid, amitrole, ammonium sulfamate, anilofos, asulam, atrazine, azimsulfuron, beflubutamid, beflubutamid-M, benazolin, benazolin-ethyl, bencarbazone, benfluralin, benfuresate, benquinotrione, bensulfuron-methyl, bensulide, bentazone, benzobicyclon, benzofenap, bicyclopyrone, bifenox, bilanafos, bipyrazone, bispyribac and its sodium salt, bixlozone, broclozone, bromacil, bromobutide, bromofenoxim, bromoxynil, bromoxynil octanoate, butachlor, butafenacil, butamifos, butralin, butroxydim, butylate, cafenstrole, carbetamide, carfentrazone-ethyl, catechin, chlomethoxyfen, chloramben, chlorbromuron, chlorflurenol-methyl, chloridazon, chlorimuron-ethyl, chlorotoluron, chlorpropham, chlorsulfuron, chlorthal-dimethyl, chlorthiamid, cinidon-ethyl, cinflubrolin, cinmethylin, cinosulfuron, clacyfos, clefoxydim, clethodim, clodinafop-propargyl, clomazone, clomeprop, clopyralid, clopyralid-olamine, cloransulam-methyl, cumyluron, cyanazine, cycloate, cyclopyranil, cyclopyrimorate, cyclosulfamuron, cycloxydim, cyhalofop-butyl, cypyrafluone, 2,4-D and its butotyl, butyl, isoctyl and isopropyl esters and its dimethylammonium, diolamine and trolamine salts, daimuron, dalapon, dalapon-sodium, dazomet, 2,4-DB and its dimethylammonium, potassium and sodium salts, desmedipham, desmetryn, dicamba and its diglycolammonium, dimethylammonium, potassium and sodium salts, dichlobenil, dichlorprop, diclofop-methyl, diclosulam, difenzoquat metilsulfate, diflufenican, diflufenzopyr, dimefuron, dimepiperate, dimepyrolimet, dimesulfazet, dimethachlor, dimethametryn, dimethenamid, dimethenamid-P, dimethipin, dimethylarsinic acid and its sodium salt, dinitramine, dinoterb, dioxopyritrione, diphenamid, diquat dibromide, dithiopyr, diuron, DNOC, endothal, EPTC, epyrifenacil, esprocarb, ethalfluralin, ethametsulfuron-methyl, ethiozin, ethofumesate, ethoxyfen, ethoxysulfuron, etobenzanid, fenoxaprop-ethyl, fenoxaprop-P-ethyl, fenoxasulfone, fenpyrazone, fenquinotrione, fentrazamide, fenuron, fenuron-TCA, feproxydim, flamprop-methyl, flamprop-M-isopropyl, flamprop-M-methyl, flazasulfuron, florasulam, florpyrauxifen, florpyrauxifen-benzyl, florpyrauxifen-methyl, fluazifop-butyl, fluazifop-P-butyl, fluazolate, flucarbazone, flucetosulfuron, fluchloralin, fluchloraminopyr, fluchloraminopyr-tefuryl, flufenazopyr, flufenacet, flufenoximacil, flufenpyr, flufenpyr-ethyl, flumetsulam, flumiclorac-pentyl, flumioxazin, fluometuron, fluoroglycofen-ethyl, flupoxam, flupyrsulfuron-methyl and its sodium salt, flurenol, flurenol-butyl, fluridone, flurochloridone, fluroxypyr, flurtamone, flusulfinam, fluthiacet-methyl, fomesafen, foramsulfuron, fosamine-ammonium, glufosinate, glufosinate-ammonium, L-glufosinate-ammonium, glufosinate-P, glyphosate and its salts such as ammonium, isopropylammonium, potassium, sodium (including sesquisodium) and trimesium (alternatively named sulfosate), halauxifen, halauxifen-methyl, halosulfuron-methyl, haloxyfop-etotyl, haloxyfop-methyl, hexazinone, hydantocidin, icafolin, icafolin-methyl, imazamethabenz-methyl, imazamox, imazapic, imazapyr, imazaquin, imazaquin-ammonium, imazethapyr, imazethapyr-ammonium, imazosulfuron, indanofan, indaziflam, indolauxipyr, indolauxipyr-cyanomethyl, iofensulfuron, iofensulfuron-sodium, iodosulfuron-methyl, iodosulfuron-sodium, ioxynil, ioxynil octanoate, ioxynil-sodium, ipfencarbazone, iptriazopyrid, isoproturon, isouron, isoxaben, isoxaflutole, isoxachlortole, lactofen, lancotrione, lancotrione-sodium, lenacil, linuron, maleic hydrazide, MCPA and its salts (e.g., MCPA-dimethylammonium, MCPA-potassium and MCPA-sodium, esters (e.g., MCPA-2-ethylhexyl, MCPA-butotyl) and thioesters (e.g., MCPA-thioethyl), MCPB and its salts (e.g., MCPB-sodium) and esters (e.g., MCPB-ethyl), mecoprop, mecoprop-P, mefenacet, mefluidide, mesosulfuron-methyl, mesotrione, metam-sodium, metamifop, metamitron, metazachlor, metazosulfuron, methabenzthiazuron, methylarsonic acid and its calcium, monoammonium, monosodium and disodium salts, methyldymron, metobenzuron, metobromuron, metolachlor, S-metolachlor, metosulam, metoxuron, metproxybicyclone, metribuzin, metsulfuron-methyl, molinate, monolinuron, naproanilide, napropamide, napropamide-M, naptalam, neburon, nicosulfuron, norflurazon, orbencarb, orthosulfamuron, oryzalin, oxadiargyl, oxadiazon, oxasulfuron, oxaziclomefone, oxyfluorfen, paraquat dichloride, pebulate, pelargonic acid, pendimethalin, penoxsulam, pentanochlor, pentoxazone, perfluidone, pethoxamid, pethoxyamid, phenmedipham, picloram, picloram-potassium, picolinafen, pinoxaden, piperophos, pretilachlor, primisulfuron-methyl, prochlorosulfone, prodiamine, profoxydim, prometon, prometryn, propachlor, propanil, propaquizafop, propazine, propham, propisochlor, propoxycarbazone, propyrisulfuron, propyzamide, prosulfocarb, prosulfuron, pyraclonil, pyraflufen-ethyl, pyraquinate, pyrasulfotole, pyrazogyl, pyrazolynate, pyrazoxyfen, pyrazosulfuron-ethyl, pyribenzoxim, pyributicarb, pyridate, pyriflubenzoxim, pyriftalid, pyriminobac-methyl, pyrimisulfan, pyrithiobac, pyrithiobac-sodium, pyroxasulfone, pyroxsulam, quinclorac, quinmerac, quinoclamine, quizalofop-ethyl, quizalofop-P-ethyl, quizalofop-P-tefuryl, rimisoxafen, rimsulfuron, saflufenacil, sethoxydim, siduron, simazine, simetryn, sulcotrione, sulfentrazone, sulfometuron-methyl, sulfosulfuron, 2,3,6-TBA, TCA, TCA-sodium, tebutam, tebuthiuron, tefuryltrione, tembotrione, tepraloxydim, terbacil, terbumeton, terbuthylazine, terbutryn, tetflupyrolimet, thenylchlor, thiazopyr, thiencarbazone, thifensulfuron-methyl, thiobencarb, tiafenacil, tiocarbazil, tolpyralate, topramezone, tralkoxydim, tri-allate, triafamone, triasulfuron, triaziflam, tribenuron-methyl, triclopyr, triclopyr-butotyl, triclopyr-triethylammonium, tridiphane, trietazine, trifloxysulfuron, trifludimoxazin, trifluralin, triflusulfuron-methyl, tripyrasulfone, tritosulfuron, vernolate, 3-(2-chloro-3,6-difluorophenyl)-4-hydroxy-1-methyl-1,5-naphthyridin-2(1H)-one, 6-chloro-4-(2,7-dimethyl-1-naphthalenyl)-5-hydroxy-2-methyl-3(2H)-pyridazinone, 5-chloro-3-[(2-hydroxy-6-oxo-1-cyclohexen-1-yl)carbonyl]-1-(4-methoxyphenyl)-2(1H)-quinoxalinone, 2-chloro-N-(1-methyl-1H-tetrazol-5-yl)-6-(trifluoromethyl)-3-pyridinecarboxamide, 7-(3,5-dichloro-4-pyridinyl)-5-(2,2-difluoroethyl)-8-hydroxypyrido[2,3-b]pyrazin-6(5H)-one), 4-(2,6-diethyl-4-methylphenyl)-5-hydroxy-2,6-dimethyl-3(2H)-pyridazinone), 5-[[(2,6-difluorophenyl)methoxy]methyl]-4,5-dihydro-5-methyl-3-(3-methyl-2-thienyl)isoxazole (previously methioxolin), 4-(4-fluorophenyl)-6-[(2-hydroxy-6-oxo-1-cyclohexen-1-yl)carbonyl]-2-methyl-1,2,4-triazine-3,5(2H,4H)-dione, 2-methyl-3-(methylsulfonyl)-N-(1-methyl-1H-tetrazol-5-yl)-4-(trifluoromethyl)benzamide, 2-methyl-N-(4-methyl-1,2,5-oxadiazol-3-yl)-3-(methylsulfinyl)-4-(trifluoromethyl)benzamide, methyl 2-[2-[2-bromo-5-[3,6-dihydro-3-methyl-2,6-dioxo-4-(trifluoromethyl)-1(2H)-pyrimidinyl]-4-fluorophenoxy]phenoxy]-2-methoxyacetate, isoxafenacil (ethyl 3-[2-chloro-5-[3,6-dihydro-3-methyl-2,6-dioxo-4-(trifluoromethyl)-1(2H)-pyrimidinyl]-4-fluorophenyl]-4,5-dihydro-5-methyl-5-isoxazolecarboxylate), 1-(2-carboxyethyl)-4-(2-pyrimidinyl)pyridazinium and its salts and esters, 5-oxo-1-[(2,3-difluorophenyl)methyl]-proline, 5-oxo-1-[(2,3,4-trifluorophenyl)methyl]-proline, L-valine, N-[2-[(2R)-1-[(2,3-difluorophenyl)methyl]-5-oxo-2-pyrrolidinyl]acetyl]-, methyl ester, L-valine, N-[2-[(2S)-1-[(2,3-difluorophenyl)methyl]-5-oxo-2-pyrrolidinyl]acetyl]-, methyl ester, cyclopropanepropanoic acid, α-[[2-[(2R)-1-[(2,3-difluorophenyl)methyl]-5-oxo-2-pyrrolidinyl]acetyl]amino]-, methyl ester, (αS)-, cyclopropanepropanoic acid, and α-[[2-[(2S)-1-[(2,3-difluorophenyl)methyl]-5-oxo-2-pyrrolidinyl]acetyl]amino]-, methyl ester, (αS)-.

Of note is a herbicide selected from the group consisting of bixlozone, broclozone, carfentrazone, carfentrazone-ethyl, chlorimuron, chlorimuron-ethyl, clomazone, florasulam, flupyrsulfuron, flupyrsulfuron-methyl, fluthiacet, fluthiacet-methyl, mesotrione, metolachlor, S-metolachlor, pinoxaden, pyroxasulfone, pyroxsulam, rimisoxafen, sulfentrazone, tembotrione, tetflupyrolimet, thifensulfuron, thifensulfuron-methyl, tribenuron and tribenuron-methyl.

The pre-polymer compounds of the present invention can also be used to microencapsulate herbicide safeners selected from the group consisting of benoxacor, cloquintocet-mexyl, cumyluron, cyometrinil, cyprosulfamide, daimuron, dichlormid, dicyclonon, dietholate, dimepiperate, fenchlorazole-ethyl, fenclorim, flurazole, fluxofenim, furilazole, isoxadifen-ethyl, mefenpyr-diethyl, mephenate, methoxyphenone, naphthalic anhydride, oxabetrinil, N-(aminocarbonyl)-2-methylbenzenesulfonamide, N-(aminocarbonyl)-2-fluorobenzenesulfonamide, 1-bromo-4-[(chloromethyl)sulfonyl]benzene (BCS), 4-(dichloroacetyl)-1-oxa-4-azospiro[4.5]decane (MON 4660), 2-(dichloromethyl)-2-methyl-1,3-dioxolane (MG 191), ethyl 1,6-dihydro-1-(2-methoxyphenyl)-6-oxo-2-phenyl-5-pyrimidinecarboxylate, 2-hydroxy-N,N-dimethyl-6-(trifluoromethyl)pyridine-3-carboxamide, 3-oxo-1-cyclohexen-1-yl 1-(3,4-dimethylphenyl)-1,6-dihydro-6-oxo-2-phenyl-5-pyrimidinecarboxylate, 2,2-dichloro-1-(2,2,5-trimethyl-3-oxazolidinyl)-ethanone and 2-methoxy-N-[[4-[[(methylamino)carbonyl]amino]phenyl]sulfonyl]-benzamide.

The pre-polymer compounds of the present invention can also be used to miciroencapsulate pheromones. Examples of pheromones include Formaldehyde; 2,2-Dibromoacetaldehyde; Acetaldehyde; 2-Methyl-2-propenal; 2-Methylpropanal; 2-Propenal; 3,3-Dibromo-2-propenal; Propanal; 2-Butenal; 2-Methyl-2-butenal; 2-Methylbutanal; 2-Methylenebutanal; 3-Methyl-2-butenal; 3-Methyl-3-butenal; 3-Methylbutanal; Butanal; (E)-2-Pentenal; 2-Methylenepentanal; 2-Pentenal; 3-Methyl-1-(vinyloxy)-butane; 4-Methylpentanal; 4-Pentenal; 5-Methylfurfural; Furan-2-carbaldehyde; Pentanal; (E)-2-Hexenal; (E)-2-Methyl-2-hexenal; (E)-3-Hexenal; (E)-4-oxo-2-Hexenal; (E,E)-2,4-Dimethyl-2,4-hexadienal; (E,E)-2,4-Hexadienal; (Z)-2-Hexenal; (Z)-3-Hexenal; (Z)-4-oxo-2-Hexenal; 1-Hexenal; 2,3-Dihydroxybenzaldehyde; 2-Hexenal; 3-((E)-2-Hexenoxy)-hexanal; 3,5-Dimethylhexanal; 3-Ethoxyhexanal; 3-Hydroxybenzaldehyde; 3-Hydroxyhexanal; 4-Hydroxy-3,5-dimethoxybenzaldehyde; 4-Hydroxybenzaldehyde; 5-Methylhexanal; Hexanal; (1R,2S,5R)-2-Methyl-5-((R)-1-oxopropan-2-yl)-cyclopentanecarbaldehyde; (1R,2S,5S)-2-Methyl-5-((R)-1-oxopropan-2-yl)-cyclopentanecarbaldehyde; (1R,5S)-6,6-Dimethylbicyclo[3.1.1]hept-2-ene-2-carbaldehyde; (1S,2S,5R)-2-Methyl-5-((R)-1-oxopropan-2-yl)-cyclopentanecarbaldehyde; (3S,8R)-2-Methyl-5-(1-formylethyl)-1-cyclopentene-1-carbaldehyde; (3S,8S)-2-Methyl-5-(1-formylethyl)-1-cyclopentene-1-carbaldehyde; (5S,8S)-2-Methyl-5-(1-formylethyl)-1-cyclopentene-1-carbaldehyde; (E)-2-(2-Hydroxyethyl)-6-methyl-2,5-heptadienal; (E)-2-(2-Hydroxyethylidene)-6-methyl-5-heptenal; (E)-2-Heptenal; (E)-2-Isopropyl-5-methyl-2-hexenal; (E)-2-Methyl-2-heptenal; (E,Z)-2,4-Heptadienal; (R)-2,6-Dimethyl-5-heptenal; (S)-4-(Prop-1-en-2-yl)-cyclohex-1-enecarbaldehyde; (Z)-2-Isopropyl-5-methyl-2-hexenal; (Z,Z)-2,4-Heptadienal; 2-(3-Methylcyclopentyl)-propanal; 2-(3-Methylcyclopentyl)-propanal; 2,3,6-Tribromo-4,5-dihydroxybenzaldehyde; 2,3-Dibromo-4,5-dihydroxybenzaldehyde; 2,6-Dimethyl-5-heptenal; 2-Methoxybenzaldehyde; 2-Methyl-1-cyclopentenecarboxaldehyde; 2-Methyl-2-heptenal; 2-Methyl-5-(1-oxopropan-2-yl)-cyclopentanecarbaldehyde; 2-Methylcyclopent-1-enecarbaldehyde; 3,3-Dimethyl-5-oxo-7-oxabicyclo[4.1.0]heptane-1-carbaldehyde; 3,4-Dimethylbenzaldehyde; 3,5-Dibromo-4,5-dihydroxybenzaldehyde; 3,5-Dibromo-4-hydroxybenzaldehyde; 3-Bromo-4,5-dihydroxybenzaldehyde; 3-Bromo-4-hydroxybenzaldehyde; 3-Bromo-5-hydroxy-4-methoxybenzaldehyde; 3-Hydroxybenzene-1,2-dicarbaldehyde; 3-Methylbenzaldehyde; 4-(Heptyloxy)-butanal; 4-Methoxybenzaldehyde; 5-(1-Formylethyl)-2-methyl-2-cyclopentene-1-carbaldehyde; 6-Methyl-5-heptenal; 6-Methylheptanal; Benzaldehyde; Cartilagineal; Cyclohexanedial; Heptanal; Taxifolial D; (1R,2S)-cis-2-Isopropenyl-1-methylcyclobutaneethanal; (1S,2R,3S)-2-(1-Formylvinyl)-5-methylcyclopentanecarbaldehyde; (1S,2S,3S)-2-(1-Formylvinyl)-5-methylcyclopentanecarbaldehyde; (2Z,6E)-8-Chloro-6-chloromethyl-2-methyl-2,6-octadienal; (4S)-(3-Oxoprop-1-en-2-yl)-cyclohex-1-enecarbaldehyde; (E)-(3,3-Dimethyl)-cyclohexylideneacetaldehyde; (E)-2-(3,3-Dimethylcyclohexylidene)-acetaldehyde; (E)-2-(4-Methyl-3-pentenyl)-butenedial; (E)-2-(4-Methyl-3-pentenylidene)-butanedial; (E)-2,7-Octadienal; (E)-2-Methyl-2-octenal; (E)-2-Methyl-5-(3-furyl)-2-pentenal; (E)-2-Octenal; (E)-3,7-Dimethyl-2,6-octadienal; (E)-3,7-Dimethyl-2,6-octadienal; (E)-3-Octenal; (E)-4-oxo-2-Octenal; (E)-7-Methyl-2-octenal; (E,E)-2,4-Octadienal; (E,E)-2,6-Dimethyl-8-hydroxy-2,6-octadienal; (E,E)-2,6-Octadienal; (E,E)-2,6-Octadienedial; (E,Z)-2,4-Octadienal; (E,Z)-2,6-Octadienal; (R)-1,2-Dimethyl-3-methylenecyclopentyl-acetaldehyde; (R)-3,7-Dimethyl-6-octenal; (Z)-(3,3-Dimethyl)-cyclohexylideneacetaldehyde; (Z)-2-(3,3-Dimethylcyclohexylidene)-acetaldehyde; (Z)-3,7-Dimethyl-2,6-octadienal; (Z,E)-3,7-Dimethyl-2,6-octadienal; 1-Octenal; 2-(1-Formylvinyl)-5-methylcyclopentanecarbaldehyde; 2-(3,4-Dihydroxyphenyl)-2-oxoacetaldehyde; 2,6,6-Trimethyl-1-cyclohexene-1-carbaldehyde; 2-Ethyloctanal; 2-Hydroxy-6-methylbenzaldehyde; 2-Methyl benzaldehyde; 2-Methyl-5-(1-formylethyl)-1-cyclopentene-1-carbaldehyde; 2-Octenal; 2-Phenylacetaldehyde; 2-Phenylpropenal; 3,4-Dihydroxyphenylglyoxal; 3,7-Dimethyl-6-octenal; 3-Ethoxy-4-hydroxybenzaldehyde; 3-Ethyl benzaldehyde; 3-Isopropyl-6-methyl benzaldehyde; 3-Octenal; 3-oxo-4-Isopropylidene-1-cyclohexene-1-carboxyaldehyde; 4-Ethylbenzaldehyde; 4-Hydroxy-2-methyl benzaldehyde; 4-Hydroxy-3-methoxybenzaldehyde; 4-Isopropenyl-1-cyclohexene-1-carbaldehyde; 4-Isopropenyl-3-oxo-1-cyclohexene-1-carboxyaldehyde; 4S-4-Isopropenyl-3-oxo-1-cyclohexene-1-carboxyaldehyde; 5-Ethylcyclopent-1-ene-carbaldehyde; 6,6-Dimethylbicyclo[3.1.1]hept-2-ene-2-carbaldehyde; 6-Methyloctanal; 7-Methyloctanal; Anisomorphal; cis-2-Isopropenyl-1-methylcyclobutaneethanal; Octanal; Peruphasmal; (1R,2S,6R)-2,6-Dimethyl-3-oxabicyclo[4.2.0]octane-2-carbaldehyde; (E)-2-Methyl-2-nonenal; (E)-2-Nonenal; (E)-3-Phenyl-2-propenal; (E)-4,8-Nonadienal; (E)-8-Methyl-2-nonenal; (E,E)-2,4-Nonadienal; (E,E,E)-2,4,6-Nonatrienal; (E,E,Z)-2,4,6-Nonatrienal; (E,Z)-2,6-Nonadienal; (E,Z,Z)-2,4,6-Nonatrienal; (Z)-2-Methyl-2-nonenal; (Z)-3-Nonenal; (Z)-4,8-Nonadienal; (Z)-4-Nonenal; (Z)-8-Methyl-2-nonenal; 2,6-Nonadienal; 2-Formyl-3-methylcyclopenteneacetaldehyde; 2-Nonenal; 2-Phenyl-2-butenal; 3-(4-Methoxyphenyl)-2-propenal; 3,5-di-tert-Butyl-4-hydroxybenzaldehyde; 3-Phenyl-2-propenal; 3-Phenylpropanal; 6-Ethyl benzaldehyde; 7,7-Dimethylbicyclo[4.1.0]hept-3-ene-3-carbaldehyde; 7-Methylnonanal; 8-Methylnonanal; 9-Acetyloxynonanal; Gibepyrone C; Nonanal; (4R,8R)-4,8-Dimethyldecanal; (4R,8S)-4,8-Dimethyldecanal; (E)-17,18,19,20-Tetranorloba-8,10,13(15)-trien-16-al; (E)-2,9-Decadienal; (E)-2-Decenal; (E)-2-Methyl-2-decenal; (E)-2-Methyl-3-(2,3-dibromo-4,5-dihydroxyphenyl)-propenal; (E)-4-oxo-2-Decenal; (E)-8-Hydroxy-4,8-dimethyl-4,9-decadienal; (E)-9-Methyl-2-decenal; (E,E)-2,4-Decadienal; (E,Z)-2,4-Decadienal; (Z)-4-Decenal; (Z)-5-Decenal; (Z)-9-Methyl-2-decenal; (Z,Z)-2,4-Decadienal; 1-Decenal; 2-Decenal; 2-Ethyldecanal; 3-(2,3-Dibromo-4,5-dihydroxyphenyl)-2-methylpropanal; 4,5-Dimethyldecanal; 4,8-Dimethyldecanal; Caraibical; Decanal; Rogiolal; (2E,4E)-2,6,10-Trimethylundeca-2,4,9-trienal; (2E,4E,7Z)-2,6,10-Trimethylundeca-2,4,7,9-tetraenal; (5E)-2,6,10-Trimethylundeca-5,9-dienal; (E)-2-Undecenal; (E)-6-Ethyl-2,10-dimethyl-5,9-undecadienal; (Z)-4-Undecenal; 10-Undecenal; 2-Butyl-2-octenal; 2-Undecenal; 3-Isopropyl-6-methyl-10-oxoundeca-2,6-dienal; 5-Methyl-2-phenyl-2-hexenal; 8-Isopropyl-5-methyl-3,4,4a,5,6,7,8,8a-octahydronaphthalene-2-carbaldehyde; Austrodoral; Oxytoxin 1; syn-4,6-Dimethylundecanal; Taxifolial A; Taxifolial B; Taxifolial C; Undecanal; (1R,6R,7S,10R)-1-Hydroxy-4-cadinen-15-al; (2R,7S,11R)-7-Acetoxy-2-hydroxynardosin-1(10)-en-12-al; (3R,5R,9R)-3,5,9-Trimethyldodecanal; (3S,6E)-7-Ethyl-3,11-dimethyldodeca-6,10-dienal; (9R)-3,5,9-Trimethyldodecanal; (E)-10-Dodecenal; (E)-2-Dodecenal; (E)-3,7,11-Trimethyl-6,10-dodecadienal; (E)-5-Dodecenal; (E)-6-Dodecenal; (E)-7-Dodecenal; (E)-8-Dodecenal; (E)-9,11-Dodecadienal; (E)-9-Dodecenal; (E,E)-3,7,11-Trimethyl-2,6,10-dodecatrienal; (E,E)-7-Ethyl-3,11-dimethyl-2,6,10-dodecatrienal; (E,E)-8,10-Dodecadienal; (E,E,E)-3,7-Dimethyl-8,11-dioxo-2,6,9-dodecatrienal; (E,E,Z)-3,7-Dimethyl-8,11-dioxo-2,6,9-dodecatrienal; (E,Z)-2,6-Dodecadienal; (E,Z)-5,7-Dodecadienal; (E,Z)-7,9-Dodecadienal; (E,Z)-8,10-Dodecadienal; (R)-10-Oxo-isodauc-3-en-15-al; (S,E)-3,7,11-Trimethyl-6,10-dodecadienal; (Z)-2-Methyl-5-((1R,5R,6S)-2,6-dimethylbicyclo[3.1.1]hept-2-en-6-yl)-pent-2-enal; (Z)-5-Dodecenal; (Z)-7-Dodecenal; (Z)-9,11-Dodecadienal; (Z)-9-Dodecenal; (Z,E)-3,7,11-Trimethyl-2,6,10-dodecatrienal; (Z,E)-5,7-Dodecadienal; (Z,E)-7-Ethyl-3,11-dimethyl-2,6,10-dodecatrienal; (Z,E)-8,10-Dodecadienal; (Z,Z)-5,7-Dodecadienal; 10-Methyldodecanal; 2,10-Dibromo-3-chloro-7-chamigrene; 2-Dodecenal; 2-Ethyldodecanal; 2-Formylguaiazulene; 3,7,11-Trimethyl-(E)-6,10-dodecadienal; 5-Hydroxy-8-methoxycalamanen-15-al; 5-Hydroxy-8-methoxycalamenene-15-al; Aplysinal; Debromoaplysinal; Dodecanal; Parahigginol D; Polygodial; Sclerosporal; Sinuketal; syn-4,6-Dimethyldodecanal; trans-Calamenen-13-al; (3R,5S,9R,7E,11E)-3,5,9,11-Tetramethyl-7,11-tridecadienal; (3S,4R,6E,10Z)-3,4,7,11-Tetramethyl-6,10-tridecadienal; (E,E)-3,5,9,11-Tetramethyltrideca-7,11-dienal; (Z)-4-Tridecenal; 13,14,15,16-Tetranorclerod-3-en-12-al; 13-Acetyloxytridecanal; 4,6-bis(4-Methylpent-3-en-1-yl)-6-methylcyclo-1,3-hexadienecarbaldehyde; Acanthodoral; Ancistrodial; Cespitulin F; Isoacanthodoral; Tridecanal; (E)-11,13-Tetradecadienal; (E)-11-Tetradecenal; (E,E)-8,10-Tetradecadienal; (E,Z)-4,9-Tetradecadienal; (E,Z)-8,10-Tetradecadienal; (Z)-11,13-Tetradecadienal; (Z)-11-Tetradecenal; (Z)-5-Tetradecenal; (Z)-7-Tetradecenal; (Z)-8-Tetradecenal; (Z)-9,13-Tetradecadien-11-ynal; (Z)-9-Tetradecenal; (Z,E)-9,11,13-Tetradecatrienal; (Z,E)-9,11-Tetradecadienal; (Z,E)-9,12-Tetradecadienal; (Z,Z)-5,8-Tetradecadienal; (Z,Z)-8,10-Tetradecadienal; (Z,Z)-9,11-Tetradecadienal; 10,12-Tetradecadienal; 2,4-Tetradecadienal; 2-Ethyltetradecanal; 3-oxo-13-Tetradecenal; 3-oxo-Tetradecanal; 5,8-Tetradecadienal; 5-Tetradecenal; Norasperenal A; Norasperenal B; Norasperenal C; Norasperenal D; Sargasal I; Sargasal II; Tetradecanal; (6R)-6-Acetoxidichotoma-3,14-diene-1,17-dial; (6R)-6-Hydroxydichotoma-3,14-diene-1,17-dial; (E,E)-3,7,11,15-Tetramethyl-6,10,14-hexadecatrienal; (E,Z)-6,8-Pentadecadienal; (E,Z)-9,11-Pentadecadienal; (E,Z,Z)-2,6,9-Pentadecatrienal; (Z)-10-Pentadecenal; (Z)-2-Chloropentadec-2-enal; (Z)-6,14-Pentadecadienal; (Z,Z)-6,9-Pentadecadienal; (Z,Z)-9,11-Pentadecadienal; 2-Hexyl-2-decenal; Azamial A; Azamial B; Isopachydictyolal; Pentadecanal; Sinularial A; Umbellacin A; Xeniafaraunol A; (1R)-Pimaral; (E)-10-Hexadecenal; (E)-11-Hexadecenal; (E)-14-Hexadecenal; (E)-14-Methyl-8-hexadecenal; (E)-3,7,11,15-Tetramethyl-2-hexadecenal; (E,E)-10,12-Hexadecadienal; (E,E)-10,14-Hexadecadienal; (E,E)-11,13-Hexadecadienal; (E,E)-9,11-Hexadecadienal; (E,E,E)-10,12,14-Hexadecatrienal; (E,E,E)-3,7,11,15-tetramethyl-2,6,10,14-hexadecatetraenal; (E,E,Z)-10,12,14-Hexadecatrienal; (E,E,Z)-4,6,11-Hexadecatrienal; (E,E,Z,Z)-4,6,11,13-Hexadecatetraenal; (E,Z)-10,12-Hexadecadienal; (E,Z)-11,13-Hexadecadienal; (E,Z)-4,6-Hexadecadienal; (E,Z)-6,11-Hexadecadienal; (E,Z)-8,11-Hexadecadienal; (E,Z)-9,11-Hexadecadienal; (R)-(E)-14-Methyl-8-hexadecenal; (R)-(Z)-14-Methyl-8-hexadecenal; (S)-(E)-14-Methyl-8-hexadecenal; (S)-(Z)-14-Methyl-8-hexadecenal; (Z)-10-Hexadecenal; (Z)-11-Hexadecenal; (Z)-12-Hexadecenal; (Z)-13-Hexadecen-11-ynal; (Z)-14-Methyl-8-hexadecenal; (Z)-3,7,11,15-Tetramethyl-2-hexadecenal; (Z)-3-oxo-9-Hexadecenal; (Z)-7-Hexadecenal; (Z)-9-Hexadecenal; (Z,E)-10,12-Hexadecadienal; (Z,E)-11,13-Hexadecadienal; (Z,E)-7,11-Hexadecadienal; (Z,E)-9,11-Hexadecadienal; (Z,Z)-10,12-Hexadecadienal; (Z,Z)-11,13-Hexadecadienal; (Z,Z)-7,10-Hexadecadienal; (Z,Z)-7,11-Hexadecadienal; (Z,Z)-9,11-Hexadecadienal; (Z,Z,E)-7,11,13-Hexadecatrienal; 11-Hexadecenal; 11-Hexadecynal; 13(16),14-Spongiadien-19-al; 2-Methylhexadecanal; 7-Hexadecenal; 9-Hexadecenal; Deacetyl-dihydro-nor-thuridillonal; Dictyodial A; Dihydro-nor-thuridillonal; Hexadecanal; Keikipukalide A; Keikipukalide B; Keikipukalide C; Keikipukalide D; Keikipukalide E; Nor-thuridillonal; Pseudoplexaural; Pukalide aldehyde; Sanadaol; (E)-2-Tridecyl-2-heptadecenal; (Z)-9-Heptadecenal; 1-Heptadecenal; 2-Heptadecenal; Globostelletin C; Globostelletin D; Heptadecanal; (E)-11-Octadecenal; (E)-13-Octadecenal; (E)-14-Octadecenal; (E)-2-Octadecenal; (E)-6-Octadecenal; (E,E)-11,14-Octadecadienal; (E,Z)-2,13-Octadecadienal; (E,Z)-3,13-Octadecadienal; (E,Z)-6,11-Octadecadienal; (Z)-11-Octadecenal; (Z)-13-Octadecenal; (Z)-9-Octadecenal; (Z,E)-13,15-Octadecadienal; (Z,Z)-11,13-Octadecadienal; (Z,Z)-13,15-Octadecadienal; (Z,Z)-3,13-Octadecadienal; (Z,Z)-9,12-Octadecadienal; (Z,Z,Z)-9,12,15-Octadecatrienal; 11-Octadecenal; 13,15-Octadecadienal; 13-Octadecenal; 16-Methyloctadecanal; 1-Octadecenal; 3,6-Dihydroxy-24-nor-9-oxo-9,11-secocholesta-7,22-dien-11-al; 9-Octadecenal; Methyloctadecanal; Octadecanal; Panicein B2; Panicein B3; Panicein C; (Z)-10-Nonadecenal; (Z)-9-Nonadecenal; 9(11)-Pargueren-16-al; Hyrtiosal; Nonadecanal; (2E,6Z,9Z)-2-Methyl-2,6,9-eicosatrienal; (Z)-11-Eicosenal; 11-Eicosenal; 12,18-di-Episcalaradial; 12b-(3′b-Hydroxybutanoyloxy)-20,24-dimethyl-24-oxo-scalara-16-en-25-al; 12b-(3′b-Hydroxypentanoyloxy)-20,24-dimethyl-24-oxo-scalara-16-en-25-al; 12-Deacetoxy-12-oxo-scalaradial; 12-Episcalaradial; 15-Eicosenal; 1-Eicosenal; 3-Deacetyl-22,23-dihydro-24,28-dehydroluffasterol B; 3-Deacetylluffasterol B; 9-Eicosenal; Anthogorgiene B; Deacetylscalaradial; Eicosadienal; Eicosanal; Mooloolabene A; Mooloolabene B; Scalaradial; and combinations thereof.

Of note as an example of pheromones include (Z)-5-decenyl acetate, dodecanyl acetate, (Z)-7-dodecenyl acetate, (E)-7-dodecenyl acetate, (Z)-8-dodecenyl acetate, (E)-8-dodecenyl acetate, (Z)-9-dodecenyl acetate, (E)-9-dodecenyl acetate, (E)-10-dodecenyl acetate, 11-dodecenyl acetate, (Z)-9,11-dodecadienyl acetate, (E)-9,11-dodecadienyl acetate, (Z)-11-tridecenyl acetate, (E)-11-tridecenyl acetate, tetradecanyl acetate, (E)-7-tetradecenyl acetate, (Z)-8-tetradecenyl acetate, (E)-8-tetradecenyl acetate, (Z)-9-tetradecenyl acetate, (E)-9-tetradecenyl acetate, (Z)-10-tetradecenyl acetate, (E)-10-tetradecenyl acetate, (Z)-11-tetradecenyl acetate, (E)-11-tetradecenyl acetate, (Z)-12-pentadecenyl acetate, (E)-12-pentadecenyl acetate, hexadecanyl acetate, (Z)-7-hexadecenyl acetate, (Z)-11-hexadecenyl acetate, (E)-11-hexadecenyl acetate, octadecanyl acetate, (E,Z)-7,9-dodecadienyl acetate, (Z,E)-7,9-dodecadienyl acetate, (E,E)-7,9-dodecadienyl acetate, (Z,Z)-7,9-dodecadienyl acetate, (E,E)-8,10-dodecadienyl acetate, (E,Z)-9,12-dodecadienyl acetate, (E,Z)-4,7-tridecadienyl acetate, (E,E)-9,11-tetradecadienyl acetate, (Z,Z)-9,12-tetradecadienyl acetate, (Z,Z)-7,11-hexadecadienyl acetate, (E,Z)-7,11-hexadecadienyl acetate, (Z,E)-7,11-hexadecadienyl acetate, (E,E)-7,11-hexadecadienyl acetate, (Z,E)-3,13-octadecadienyl acetate, (E,Z)-3,13-octadecadienyl acetate, (E,E)-3,13-octadecadienyl acetate, decanol, (Z)-6-nonenol, (E)-6-nonenol, dodecanol, (Z)-5-decenol, 11-dodecenol, (Z)-7-dodecenol, (E)-7-dodecenol, (Z)-8-dodecenol, (E)-8-dodecenol, (E)-9-dodecenol, (Z)-9-dodecenol, (E)-9,11-dodecadienol, (Z)-9,11-dodecadienol, (Z,E)-5,7-dodecadienol, (E,E)-5,7-dodecadienol, (E,E)-8,10-dodecadien-1-ol, (E,Z)-8,10-dodecadienol, (Z,Z)-8,10-dodecadienol, (Z,E)-8,10-dodecadienol, (E,Z)-7,9-dodecadienol, (Z,Z)-7,9-dodecadienol, (E)-5-tetradecenol, (Z)-8-tetradecenol, (Z)-9-tetradecenol, (E)-9-tetradecenol, (Z)-10-tetradecenol, (Z)-11-tetradecenol, (E)-11-tetradecenol, (Z)-11-hexadecenol, (Z,E)-9,11-tetradecadienol, (Z,E)-9,12-tetradecadienol, (Z,Z)-9,12-tetradecadienol, (Z, Z)-10,12-tetradecadienol, (Z,Z)-7,11-hexadecadienol, (Z,E)-7,11-hexadecadienol, (E)-14-methyl-8-hexadecen-1-ol, (Z)-14-methyl-8-hexadecen-1-ol, (E,E)-10,12-hexadecadienol, (E,Z)-10,12-hexadecadienol, dodecanal, (Z)-9-dodecanal, tetradecanal, (Z)-7-tetradecenal, (Z)-9-tetradecenal, (Z)-11-tetradecenal, (E)-11-tetradecenal, (E)-11,13-tetradecadienal, (E,E)-8,10-tetradecadienal, (Z,E)-9,11-tetradecadienal, (Z,E)-9,12-tetradecadienal, hexadecanal, (Z)-8-hexadecenal, (Z)-9-hexadecenal, (Z)-10-hexadecenal, (E)-10-hexadecenal, (Z)-11-hexadecenal, (E)-hexadecenal, (Z)-12-hexadecenal, (Z)-13-hexadecenal, (Z)-14-methyl-8-hexadecenal, (E)-14-methyl-8-hexadecenal, (Z,Z)-7,11-hexadecadienal, (Z,E)-7,11-hexadecadienal, (Z,E)-9,11-hexadecadienal, (E,E)-10,12-hexadecadienal, (E,Z)-10,12-hexadecadienal, (Z,E)-10,12-hexadecadienal, (Z,Z)-10,12-hexadecadienal, (Z,Z)-11,13-hexadecadienal, octadecenal, (Z)-11-octadecenal, (E)-13-octadecenal, (Z)-13-octadecenal, (Z)-5-decenyl 3-methylbutanoate, (+) cis-7,8-epoxy-2-methyloctadecane.

Also of note as examples of pheromones include citral; geranial; neral; tetradecan-1-al; pentadecan-1-al; pentadecen-1-al; hexadecan-1-al; (Z)-9-hexadecen-1-al; (Z)-11-hexadecen-1-al; (7E,9E)-undeca-7,9-dien-1-al; (11Z, 13Z)-hexadecadien-1-al; (9Z,12E)-tetradecadien-1-al; (8E,10E)-dodecadien-1-al; (11Z)-hexadecadien-1-al; (9Z)-tetradecen-1-al; 6,10-dimethyl-5,9-undecadien-2-ol; (6E)-7,11-dimethyl-3-methylene-1,6,10-dodecatriene; [1S-(1a,2b,5 a)]-4,6,6-trimethyl-Bicyclo[3.1.1]hept-3-en-2-ol; 10-Hexadecenal; (Z)-10-hexadecenal; (E)-10-hexadecenal; and combinations thereof.

The pre-polymer compounds of the present invention can also be used to microencapsulate insecticides. Examples of insecticides include abamectin, acephate, acequinocyl, acetamiprid, acrinathrin, acynonapyr, afidopyropen ([(3S,4R,4aR,6S,6aS,12R,12aS,12bS)-3-[(cyclopropylcarbonyl)oxy]-1,3,4,4a,5,6,6a,12,12a,12b-decahydro-6,12-dihydroxy-4,6a,12b-trimethyl-11-oxo-9-(3-pyridinyl)-2H, 11H-naphtho[2,1-b]pyrano[3,4-e]pyran-4-yl]methyl cyclopropanecarboxylate), amidoflumet, amitraz, avermectin, azadirachtin, azinphos-methyl, benfuracarb, bensultap, benzpyrimoxan, bifenthrin, kappa-bifenthrin, bifenazate, bistrifluron, borate, broflanilide, buprofezin, cadusafos, carbaryl, carbofuran, cartap, carzol, chlorfenapyr, chlorfluazuron, chloroprallethrin, chlorpyrifos, chlorpyrifos-e, chlorpyrifos-methyl, chromafenozide, clofentezin, chloroprallethrin, clothianidin, cycloprothrin, cycloxaprid ((5S,8R)-1-[(6-chloro-3-pyridinyl)methyl]-2,3,5,6,7,8-hexahydro-9-nitro-5,8-Epoxy-1H-imidazo[1,2-a]azepine), cyenopyrafen, cyflumetofen, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, diafenthiuron, diazinon, dicloromesotiaz, dieldrin, diflubenzuron, dimefluthrin, dimehypo, dimethoate, dimpropyridaz, dinotefuran, diofenolan, emamectin, emamectin benzoate, endosulfan, esfenvalerate, ethiprole, etofenprox, epsilon-metofluthrin, etoxazole, fenbutatin oxide, fenitrothion, fenothiocarb, fenoxycarb, fenpropathrin, fenvalerate, fipronil, flometoquin (2-ethyl-3,7-dimethyl-6-[4-(trifluoromethoxy)phenoxy]-4-quinolinyl methyl carbonate), flonicamid, fluazaindolizine, flucythrinate, flufenerim, flufenoxuron, flufenoxystrobin(methyl (αE)-2-[[2-chloro-4-(trifluoromethyl)phenoxy]methyl]-α-(methoxymethylene)benzeneacetate), fluensulfone (5-chloro-2-[(3,4,4-trifluoro-3-buten-1-yl)sulfonyl]thiazole), fluhexafon, fluopyram, flupiprole (1-[2,6-dichloro-4-(trifluoromethyl)phenyl]-5-[(2-methyl-2-propen-1-yl)amino]-4-[(trifluoromethyl)sulfinyl]-1H-pyrazole-3-carbonitrile), flupyradifurone (4-[[(6-chloro-3-pyridinyl)methyl](2,2-difluoroethyl)amino]-2(5H)-furanone), flupyrimin, fluvalinate, tau-fluvalinate, fluxametamide, fonophos, formetanate, fosthiazate, gamma-cyhalothrin, halofenozide, heptafluthrin ([2,3,5,6-tetrafluoro-4-(methoxymethyl)phenyl]methyl 2,2-dimethyl-3-[(1Z)-3,3,3-trifluoro-1-propen-1-yl]cyclopropanecarboxylate), hexaflumuron, hexythiazox, hydramethylnon, imidacloprid, indoxacarb, insecticidal soaps, isofenphos, isocycloseram, kappa-tefluthrin, lambda-cyhalothrin, lufenuron, malathion, meperfluthrin ([2,3,5,6-tetrafluoro-4-(methoxymethyl)phenyl]methyl (1R,3S)-3-(2,2-dichloroethenyl)-2,2-dimethylcyclopropanecarboxylate), metaflumizone, metaldehyde, methamidophos, methidathion, methiocarb, methomyl, methoprene, methoxychlor, metofluthrin, methoxyfenozide, epsilon-metofluthrin, epsilon-momfluorothrin, monocrotophos, monofluorothrin ([2,3,5,6-tetrafluoro-4-(methoxymethyl)phenyl]methyl 3-(2-cyano-1-propen-1-yl)-2,2-dimethylcyclopropanecarboxylate), nicotine, nitenpyram, nithiazine, novaluron, noviflumuron, N-[1,1-dimethyl-2-(methylthio)ethyl]-7-fluoro-2-(3-pyridinyl)-2H-indazole-4-carboxamide, N-[1,1-dimethyl-2-(methylsulfinyl)ethyl]-7-fluoro-2-(3-pyridinyl)-2H-indazole-4-carboxamide, N-[1,1-dimethyl-2-(methylsulfonyl)ethyl]-7-fluoro-2-(3-pyridinyl)-2H-indazole-4-carboxamide, N-(1-methylcyclopropyl)-2-(3-pyridinyl)-2H-indazole-4-carboxamide, N-[1-(difluoromethyl)cyclopropyl]-2-(3-pyridinyl)-2H-indazole-4-carboxamide, oxamyl, oxazosulfyl, parathion, parathion-methyl, permethrin, phorate, phosalone, phosmet, phosphamidon, pirimicarb, profenofos, profluthrin, propargite, protrifenbute, pyflubumide (1,3,5-trimethyl-N-(2-methyl-1-oxopropyl)-N-[3-(2-methylpropyl)-4-[2,2,2-trifluoro-1-methoxy-1-(trifluoromethyl)ethyl]phenyl]-1H-pyrazole-4-carboxamide), pymetrozine, pyrafluprole, pyrethrin, pyridaben, pyridalyl, pyrifluquinazon, pyriminostrobin(methyl (αE)-2-[[[2-[(2,4-dichlorophenyl)amino]-6-(trifluoromethyl)-4-pyrimidinyl]oxy]methyl]-α-(methoxymethylene)benzeneacetate), pyriprole, pyriproxyfen, rotenone, ryanodine, silafluofen, spinetoram, spinosad, spirodiclofen, spiromesifen, spiropidion, spirotetramat, sulprofos, sulfiflumin, sulfoxaflor (N-[methyloxido[1-[6-(trifluoromethyl)-3-pyridinyl]ethyl]-λ⁴-sulfanylidene]cyanamide), tebufenozide, tebufenpyrad, teflubenzuron, tefluthrin, kappa-tefluthrin, terbufos, tetrachlorvinphos, tetramethrin, tetramethylfluthrin ([2,3,5,6-tetrafluoro-4-(methoxymethyl)phenyl]methyl 2,2,3,3-tetramethylcyclopropanecarboxylate), thiacloprid, thiamethoxam, thiodicarb, thiosultap-sodium, tiapyrachlor, tioxazafen (3-phenyl-5-(2-thienyl)-1,2,4-oxadiazole), tolfenpyrad, tralomethrin, triazamate, trichlorfon, triflumezopyrim (2,4-dioxo-1-(5-pyrimidinylmethyl)-3-[3-(trifluoromethyl)phenyl]-2H-pyrido[1,2-a]pyrimidinium inner salt), triflumuron, tyclopyrazoflor, zeta-cypermethrin, Bacillus thuringiensis delta-endotoxins, entomopathogenic bacteria, entomopathogenic viruses or entomopathogenic fungi, can combinations thereof.

Examples of insecticides also include diamides, such as chlorantraniliprole, cyantraniliprole, tetrachlorantraniliprole, bromoantraniliprole, dichlorantraniliprole, tetraniliprole, cyclaniliprole, cyhalodiamide, and flubendiamide

The pre-polymer compounds of the present invention can also be used to microencapsulate fungicides. Examples of fungicides include fungicides such as acibenzolar-S-methyl, aldimorph, ametoctradin, aminopyrifen, amisulbrom, anilazine, azaconazole, azoxystrobin, benalaxyl (including benalaxyl-M), benodanil, benomyl, benthiavalicarb (including benthiavalicarb-isopropyl), benzovindiflupyr, bethoxazin, binapacryl, biphenyl, bifemetstrobin, bitertanol, bixafen, blasticidin-S, boscalid, bromuconazole, bupirimate, buthiobate, carboxin, carpropamid, captafol, captan, carbendazim, chloroneb, chlorothalonil, chlozolinate, copper hydroxide, copper oxychloride, copper sulfate, coumoxystrobin, cyazofamid, cyflufenamid, cymoxanil, cyproconazole, cyprodinil, dichlobentiazox, dichlofluanid, diclocymet, diclomezine, dicloran, diethofencarb, difenoconazole, diflumetorim, dimethirimol, dimethomorph, dimoxystrobin, diniconazole (including diniconazole-M), dinocap, dipymetitrone, dithianon, dithiolanes, dodemorph, dodine, econazole, etaconazole, edifenphos, enoxastrobin (also known as enestroburin), epoxiconazole, ethaboxam, ethirimol, etridiazole, famoxadone, fenamidone, fenaminstrobin, fenarimol, fenbuconazole, fenfuram, fenhexamide, fenoxanil, fenpiclonil, fenpicoxamid, fenpropidin, fenpropimorph, fenpyrazamine, fentin acetate, fentin hydroxide, ferbam, ferimzone, flometoquin, florylpicoxamid, fluopimomide, fluazinam, fludioxonil, flufenoxystrobin, fluindapyr, flumorph, fluopicolide, fluopyram, fluoxapiprolin, fluoxastrobin, fluquinconazole, flusilazole, flusulfamide, flutianil, flutolanil, flutriafol, fluxapyroxad, folpet, fthalide (also known as phthalide), fuberidazole, furalaxyl, furametpyr, hexaconazole, hymexazole, guazatine, imazalil, imibenconazole, iminoctadine albesilate, iminoctadine triacetate, inpyrfluxam, iodicarb, ipconazole, ipfentrifluconazole, ipflufenoquin, isofetamid, iprobenfos, iprodione, iprovalicarb, isoflucypram, isoprothiolane, isopyrazam, isotianil, kasugamycin, kresoxim-methyl, lancotrione, mancozeb, mandipropamid, mandestrobin, maneb, mapanipyrin, mefentrifluconazole, mepronil, meptyldinocap, metalaxyl (including metalaxyl-M/mefenoxam), metconazole, methasulfocarb, metiram, metominostrobin, metyltetraprole, metrafenone, myclobutanil, naftitine, neo-asozin (ferric methanearsonate), nuarimol, octhilinone, ofurace, orysastrobin, oxadixyl, oxathiapiprolin, oxolinic acid, oxpoconazole, oxycarboxin, oxytetracycline, penconazole, pencycuron, penflufen, penthiopyrad, perfurazoate, phosphorous acid (including salts thereof, e.g., fosetyl-aluminm), picoxystrobin, piperalin, polyoxin, probenazole, prochloraz, procymidone, propamocarb, propiconazole, propineb, proquinazid, prothiocarb, prothioconazole, pydiflumetofen (Adepidyn®), pyraclostrobin, pyrametostrobin, pyrapropoyne, pyraoxystrobin, pyraziflumid, pyrazophos, pyribencarb, pyributacarb, pyridachlometyl, pyrifenox, pyriofenone, perisoxazole, pyrimethanil, pyrifenox, pyrrolnitrin, pyroquilon, quinaminoprole, quinconazole, quinmethionate, quinofumelin, quinoxyfen, quintozene, silthiofam, sedaxane, simeconazole, spiroxamine, streptomycin, sulfur, tebuconazole, tebufloquin, teclofthalam, tecloftalam, tecnazene, terbinafine, tetraconazole, thiabendazole, thifluzamide, thiophanate, thiophanate-methyl, thiram, tiadinil, tolclofos-methyl, tolprocarb, tolyfluanid, triadimefon, triadimenol, triarimol, triazoxide, tribasic copper sulfate, triclopyricarb, tridemorph, trifloxystrobin, triflumizole, trimoprhamide tricyclazole, trifloxystrobin, triforine, triticonazole, uniconazole, validamycin, valifenalate (also known as valifenal), vinclozolin, zineb, ziram, zoxamide, 1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone, and combinations thereof.

The pre-polymer compounds of the present invention can also be used to microencapsulate nematocides. Examples of nematocides include fluopyram, spirotetramat, thiodicarb, fosthiazate, abamectin, iprodione, fluensulfone, dimethyl disulfide, tioxazafen, 1,3-dichloropropene (1,3-D), metam (sodium and potassium), dazomet, chloropicrin, fenamiphos, ethoprophos, cadusaphos, terbufos, imicyafos, oxamyl, carbofuran, tioxazafen, Bacillus firmus, Pasteuria nishizawae, and combinations thereof. An example of a bactericide is streptomycin. Examples of acaricides include amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben, tebufenpyrad, and combinations thereof.

Of note is a composition comprising a compound of the invention (in an agriculturally effective amount), at least one additional active ingredient selected from the group consisting of other herbicides and herbicide safeners (in an effective amount), and at least one component selected from the group consisting of surfactants, solid diluents and liquid diluents.

The following abbreviations are used in the Index Tables which follow: (R) or (S) denotes the absolute chirality of the asymmetric carbon center. The abbreviation “Cmpd. No.” stands for “Compound Number”. The abbreviation “Ex.” stands for “Example” and is followed by a number indicating in which Synthesis Example example the compound is prepared. Mass spectra are reported with an estimated precision within ±0.5 Da as the molecular weight of the highest isotopic abundance parent ion (M+1) formed by addition of H⁺ (molecular weight of 1) to the molecule observed by using atmospheric pressure chemical ionization (AP+).

INDEX TABLE A

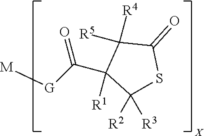

1
R² is H, R³ is H, R⁴ is H, R⁵ is H; and

| Cmpd. No. | X | M | G | R¹ | Mass (M + 1) |
|---|---|---|---|---|---|
| 1 | 2 | M-7 | O,O | H | 395 |
| 2 (Ex. 1) | 2 | M-5 | O,O | H | 381 |
| 3 (Ex. 5) | 2 | M-9 | O,O | H | 455 |
| 4 | 2 | M-12 | O,O | H | 455 |
| 5 | 2 | M-3 | O,O | H | 396 |
| 6 (Ex. 6) | 2 | M-8 | O,O | H | 385 |
| 7 (Ex. 7) | 3 | M-6 | O,O,O | H | 646 |
| 8 | 2 | M-15 | O,O | H | 477 |
| 9 | 2 | M-10 | O,O | H | 376 |
| 10 | 2 | M-11 | O,O | H | 347 |
| 11 | 2 | M-1 | O,O | H | 319 |
| 12 (Ex. 10) | 2 | M-2 | O,O | H | 331 |
| 13 | 2 | M-2 | NH,O | H | 332 |
| 14 | 2 | M-4 | O,O | H | 347 |
| 15 (Ex. 9) | 2 | M-13 | O,O | H | 395 |
| 16 | 2 | M-14 | NH,O | H | 394 |
| 17 | 2 | M-16 | O,O | H | 471 |
| 18 | 3 | M-17 | O,O,O | H | 477 |
| 19 | 2 | M-17 | O,O | H | 439 |
| 20 | 4 | M-26 | O,O,O,O | H | 777 |
| 21 | 3 | M-17 | O,NH,O | H | 476 |
| 22 (Ex. 1) | 2 | M-24 | O,O | H | 452 |
| 23 (Ex. 8) | 3 | M-23 | O,O,O | H | 519 |
| 24 (Ex. 2) | 3 | M-25 | O,O,O | H | 567 |
| 25 (Ex. 3) | 2 | M-19 | O,O | H | 403 |
| 26 | 2 | M-20 | O,O | H | 403 |
| 27 | 2 | M-21 | O,O | H | 456 |
| 28 | 2 | M-26 | O,O | H | 347 |
| 29 | 2 | M-30 | O,NH | H | 452 |
| 30 | 2 | M-31 | O,O | H | * |
| 31 | 2 | M-32 | O,O | H | 452 |
| 32 | 2 | M-33 | O,O | H | 481 |
| 33 | 2 | M-34 | O,O | H | 487 |
| 34 | 2 | M-35 | O,O | H | 471 |
| 35 | 2 | M-36 | O,O | H | 533 |
| 36 (Ex. 4) | 2 | M-37 | O,O | H | 495 |
| 37 | 2 | M-39 | O,O | H | 533 |
| 38 | 2 | M-40 | O,O | H | 592 |
| 39 | 2 | M-38 | O,O | H | 460 (M − 1) |
| 40 | 2 | M-41 | O,O | H | 466 |
| 41 | 2 | M-42 | O,O | H | 490 |
| 42 | 3 | M-43 | O,O,O | H | * |
| 43 | 3 | M-44 | O,O,O | H | 610 |
| 44 | 3 | M-45 | O,O,O | H | 644 (M − H) |
| 45 | 3 | M-46 | O,O,O | H | 633 |
| 46 | 2 | M-14 | NH,O | H | 394 |
| 47 | 2 | M-47 | O,O | H | 543 |
| 48 | 3 | M-17 | NH,O,O | H | 476 |
| 49 | 3 | M-17 | O,NH,NH | H | 475 |
| 50 | 2 | M-13 | NH,O | H | 394 |
| 51 | 2 | M-29 | O,O | H | 459 |

*See Index Table B for ¹H NMR data.
**See Synthesis Example for ¹H NMR data.

INDEX TABLE B

| Cmpd. No. | ¹H NMR Data (CDCl₃ solution, 500 MHz unless indicated otherwise)^{a} |
|---|---|
| 30 | δ ppm 1.44 (s , 9H), 2.74-279 (m, 2H), 2.89-2.96 (m, 2H), 3.32-3.25 (brs, 1H), 3.41-3.44 (m, |
| | 3H), 3.58-3.68 (m, 4H), 4.23 (br s, 1H), 4.45 br s, 1H), 4.72 (br s, 1H), 5.18 (br s, 1H). |
| 42 | δ ppm 1.44 (s, 9H), 2.72-2.83 (m, 3H), 2.86-2.97 (m, 3H), 3.39-3.52 (m, 3H), 3.62 (s, 6H), |
| | 4.20-4.49 (m, 6H), 4.88 (br s, 1H). |

^{a}¹H NMR data are in ppm downfield from tetramethylsilane. Couplings are designated by (s)-singlet, doublet, (m)-multiplet, (br s)-broad singlet.

### EXAMPLES OF THE INVENTION

### Example 1: Preparation of a Blank Polymer

Blank microencapsulation formulations were prepared using a pre-polymer of the invention (see Index Table A). To prepare the blank microencapsulation polymer, a pre-polymer of the invention and trimethylolpropane triacrylate (TMPTA) were dissolved in Aromatic 200ND and this constituted the organic phase. Water, Reax 88B, Xiameter AFE 100, Mowiol 8-88 (10% aqueous solution) and Chitosan (0.5% aqueos solution) were mixed and heated to 70° C. The organic phase was added to the aqueous phase and emulsified. Upon adding 1,6-hexamethylenediamine (HMDA) in the aqueous phase instantaneous reaction occurs at the organic droplet interface between the pre-polymer of the invention, trimethylolpropane triacrylate, and HMDA leading to stable capsule wall formation. Blank microcapsules slurry was centrifuged to wash out any components of an aqueous phase, and optionally washed with a water-immiscible solvent. Then the remaining polymer was filtered, dried and collected for testing.

TABLE 1
Blank formulation(s)

| Ingredients | Amount (wt. %) |
|---|---|
| Aromatic 200ND | 30.0 |
| Reax 88B | 1.5 |
| Mowiol 8-88 | 6.0 |
| (10% aqueous solution) | |
| Xiameter AFE 100 | 0.1 |
| pre-polymer Compound | 5 |
| HMDA (43% aqueous solution) | 2.9 |
| Trimethylolpropane triacrylate | 2.1 |
| Chitosan (0.5% aqueous solution) | 4.8 |
| Water | 47.7 |
| Total | 100 |

### Example 2: Preparation of Microencapsulated Bixolozone Formulations

A microencapsulation formulation for an active ingredient bixlozone using a pre-polymer compound of the invention. To prepare this formulation, bixlozone technical was dissolved in Jeffsol AG-1705 at 70° C. Then, Compound 18, a pre-polymer compound of the present invention, and TMPTA were added. This constituted the organic phase. Water, Reax 88B, Xiameter AFE 100, Mowiol 8-88 (10% solution) and Chitosan (0.5% solution) were mixed and heated to 70° C. This constituted the aqueous phase. For the microencapsulation process, the aqueous phase was charged to a microencapsulation reactor under slow stirring, the organic phase was added, and the stirring was increased to 5000 rpm for 120 s to form an oil-in-water emulsion. The HMDA (aqueous solution) was added at once to the emulsion under stirring, and an instantaneous reaction between the pre-polymer compound, TMPTA and the HMDA afforded microencapsule wall formation around the organic droplets via interfacial polymerization. The microcapsule suspension was cured at 60° C. for 6 h. Salts, xanthan gum, and pH adjusters were added at the end to complete the process. For pre-polymer Compound 23 and Compound 15, the same procedure was followed for Compound 18 with variations in acrylate and amine percentages based off equivalency weights calculated according to the following Tables.

TABLE 2
Bixlozone Capsule Formulation with Compound 18

| Ingredients | Amount (Wt. %) |
|---|---|
| Bixlozone | 20.00 |
| Jeffsol AG-1705 | 15.00 |
| Compound 18 | 5.00 |
| Trimethylolpropane triacrylate (TMPTA) | 3.11 |
| 4-methoxyphenol | 0.05 |
| Reax 88B | 1.25 |
| Mowiol 8-88 (10% aqueous solution) | 5.00 |
| Chitosan (0.5%) | 4.00 |
| Xiameter AFE 100 | 0.05 |
| Sodium nitrate | 4.00 |
| Calcium chloride | 4.00 |
| 2% Kelzan AP/AS + 1% Proxel GXL | 1.50 |
| (aqueous solution) | |
| HMDA (43% aqueous solution) | 4.20 |
| Water | 37.04 |
| Total | 100.00 |

TABLE 3
Bixlozone Capsules formulation with Compound 15

| Ingredients | Amount (Wt. %) |
|---|---|
| bixlozone | 20.00 |
| Jeffsol AG-1705 | 15.00 |
| Compound 15 | 5.00 |
| Trimethylolpropane triacrylate (TMPTA) | 2.50 |
| 4-methoxyphenol | 0.05 |
| Reax 88B | 1.25 |
| Mowiol 8-88 (10% aqueous solution) | 5.00 |
| Chitosan (0.5% aqueous solution) | 4.00 |
| Xiameter AFE 100 | 0.05 |
| Sodium nitrate | 4.00 |
| Calcium chloride | 4.00 |
| 2% Kelzan AP/AS + 1% Proxel GXL | 1.50 |
| (aqeuous solution) | |
| HMDA (43% aqueous solution) | 3.43 |
| Water | 34.22 |
| Total | 100.00 |

TABLE 4
Bixlozone Capsule Formulation with Compound 23

| Ingredients | Amount (Wt. %) |
|---|---|
| bixlozone | 20.00 |
| Jeffsol AG-1705 | 15.00 |
| Compound 23 | 5.00 |
| Trimethylolpropane triacrylate (TMPTA) | 2.86 |
| 4-methoxyphenol | 0.05 |
| Reax 88B | 1.25 |
| Mowiol 8-88 (10% aqueous solution) | 5.00 |
| Chitosan (0.5% aqeuous solution) | 4.00 |
| Xiameter AFE 100 | 0.05 |
| Sodium nitrate | 4.00 |
| Calcium chloride | 4.00 |
| 2% Kelzan AP/AS + 1% Proxel GXL | 1.50 |
| (aqeuous solution) | |
| HMDA (43% aqueous solution) | 3.90 |
| Water | 37.29 |
| Total | 100.00 |

### Example 3: Preparation of Microencapsulated Clomazone Formulations

A microencapsulation formulation was prepared with the active ingredient clomozone using a pre-polymer compound. To prepare this formulation, clomazone technical was dissolved in Jeffsol AG 1705 at 70° C. Then Compound 18, a pre-polymer compound of the present invention, and TMPTA were added. This constituted the organic phase. Water, Reax 88B, Xiameter AFE 100, Mowiol 8-88 (10% aqueous solution) and Chitosan (0.5% aqueous solution) were mixed and heated to 70° C. This constituted the aqueous phase. For the microencapsulation process, the aqueous phase was charged to a microencapsulation reactor under slow stirring, the organic phase was added, and the stirring was increased to 5000 rpm for 120 s to form an oil-in-water emulsion. The HMDA was added at once to the emulsion under stirring, and an instantaneous reaction between the pre-polymer compound, TMPTA and the amine afforded microencapsule wall formation around the organic droplets via interfacial polymerization. The capsule suspension was cured at 60° C. for 6 h. Salts, xanthan gum, and pH adjusters were added at the end to complete the process. For pre-polymer Compound 23 and Compound 15 the same procedure was followed for Compound 18 with variations in acrylate and amine percentage based off equivalency weights calculated according to the following Tables.

TABLE 5
Clomazone Capsule Formulation with Compound 18

| Ingredients | Amount (Wt. %) |
|---|---|
| Clomazone | 20.00 |
| Jeffsol AG-1705 | 15.00 |
| Compound 18 | 5.00 |
| Trimethylolpropane triacrylate (TMPTA) | 3.11 |
| 4-methoxyphenol | 0.05 |
| Reax 88B | 1.25 |
| Mowiol 8-88 (10% aqueous solution) | 5.00 |
| Chitosan (0.5% aqueous solution) | 4.00 |
| Xiameter AFE 100 | 0.05 |
| Sodium nitrate | 4.00 |
| Calcium chloride | 4.00 |
| 2% Kelzan AP/AS + 1% Proxel GXL | 1.50 |
| (aqueous solution) | |
| HMDA (43% aqueous solution) | 4.20 |
| Water | 37.04 |
| Total | 100.00 |

TABLE 6
Clomazone Capsule Formulation with Compound 15

| Ingredients | Amount (Wt. %) |
|---|---|
| Clomazone | 20.00 |
| Jeffsol AG-1705 | 15.00 |
| Compound 15 | 5.00 |
| TMPTA | 2.50 |
| 4-methoxyphenol | 0.05 |
| Reax 88B | 1.25 |
| Mowiol 8-88 (10% aqueous solution) | 5.00 |
| Chitosan (0.5% aqueous solution) | 4.00 |
| Xiameter AFE 100 | 0.05 |
| Sodium nitrate | 4.00 |
| Calcium chloride | 4.00 |
| 2% Kelzan AP/AS + 1% Proxel GXL | 1.50 |
| (aqueous solution) | |
| HMDA (43% aqueous solution) | 3.43 |
| Water | 34.22 |
| Total | 100.00 |

TABLE 7
Clomazone Capsule Formulation with Compound 23

| Ingredients | Amount (Wt. %) |
|---|---|
| clomazone | 20.00 |
| Jeffsol AG-1705 | 15.00 |
| Compound 23 | 5.00 |
| TMPTA | 2.86 |
| 4-methoxyphenol | 0.05 |
| Reax 88B | 1.25 |
| Mowiol 8-88 (10% aqueous solution) | 5.00 |
| Chitosan (0.5% aqeuous solution) | 4.00 |
| Xiameter AFE 100 | 0.05 |
| Sodium nitrate | 4.00 |
| Calcium chloride | 4.00 |
| 2% Kelzan AP/AS + 1% Proxel GXL | 1.50 |
| (aqueous solution) | |
| HMDA (43% aqueous solution) | 3.90 |
| Water | 37.29 |
| Total | 100.00 |

What is claimed is:

1. A compound selected from Formula 1, and stereoisomers thereof, 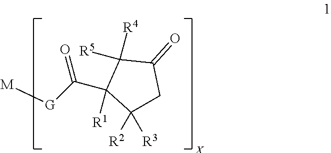
wherein:
X is 2, 3 or 4;
M is a di-, tri- or tetra-valent core moiety;
each G is independently O or NR⁶;
R¹ is hydrogen, C₁-C₄ alkyl or C₃-C₆ cycloalkyl;
R² is hydrogen, C₁-C₄ alkyl or C₃-C₆ cycloalkyl;
R³ is hydrogen, C₁-C₄ alkyl or C₃-C₆ cycloalkyl;
R⁴ is hydrogen, C₁-C₄ alkyl or C₃-C₆ cycloalkyl;
R⁵ is hydrogen, C₁-C₄ alkyl or C₃-C₆ cycloalkyl; and
R⁶ is hydrogen or C₁-C₄ alkyl.

2. The compound of claim 1 wherein M is selected from the group consisting of

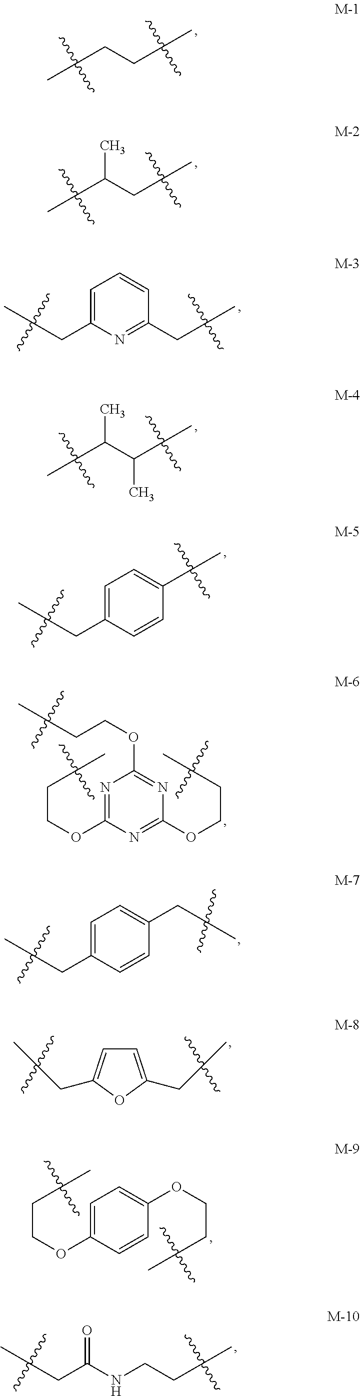

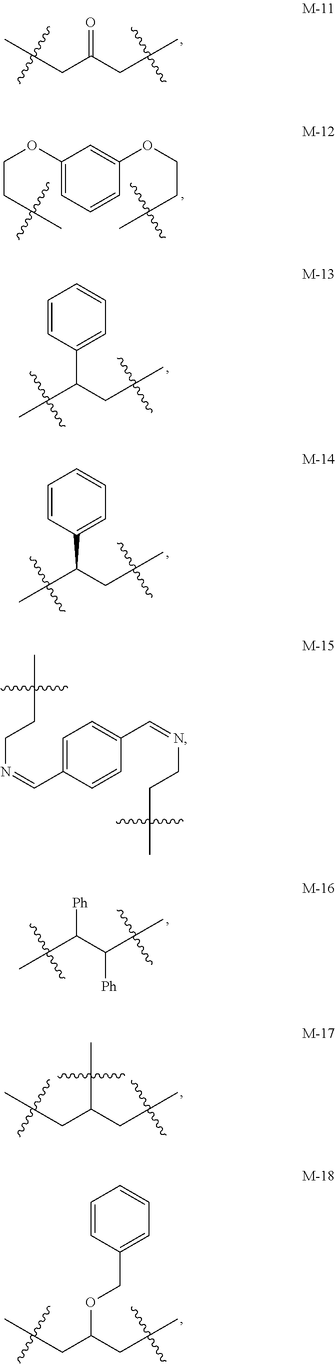

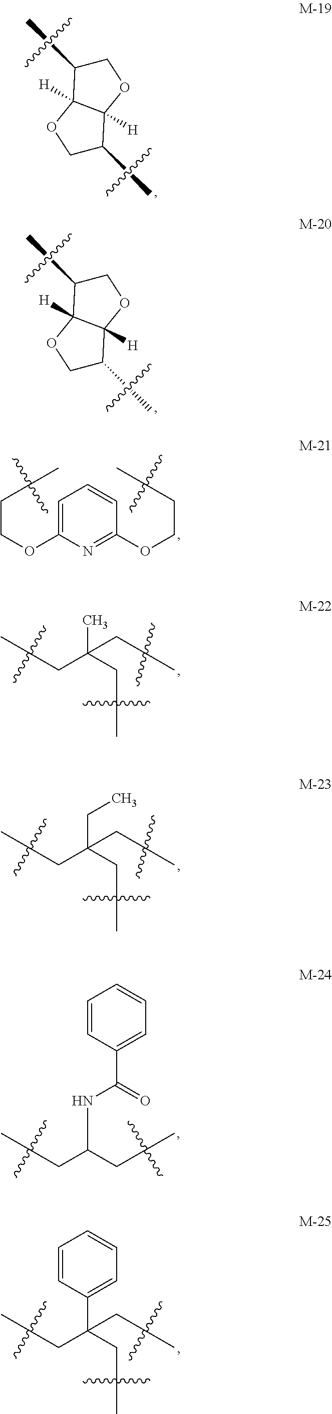

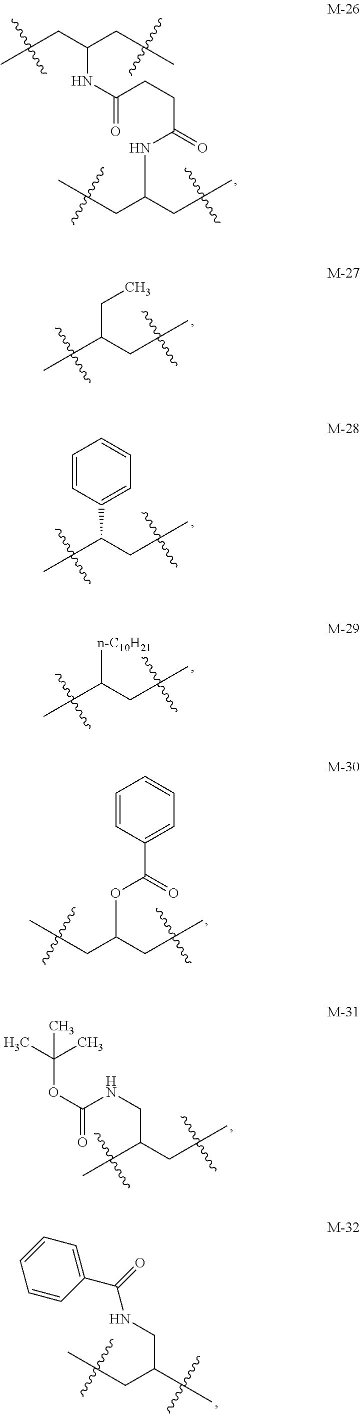

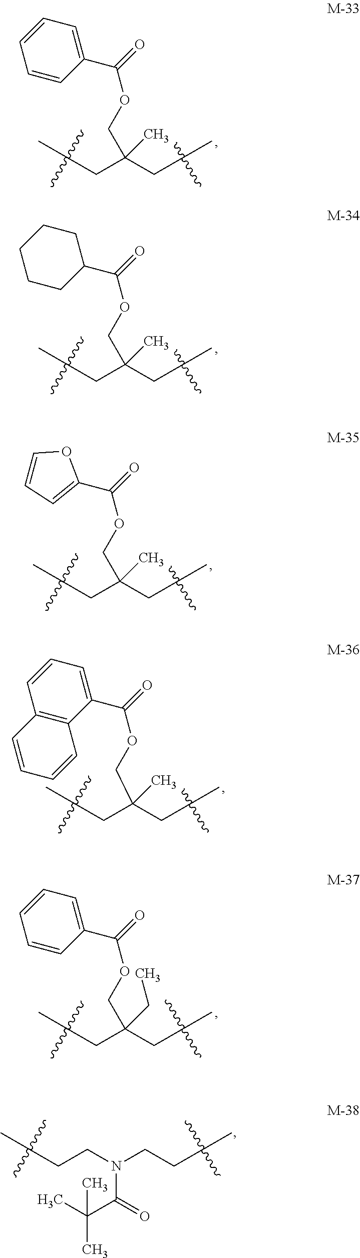 [SMILES: *CC(C)(C*)COC(=O)c1ccccc1 ; *CC(C)(C*)COC(=O)C1CCCCC1 ; *CC(C)(C*)COC(=O)c1ccco1 ; *CC(C)(C*)COC(=O)c1cccc2ccccc12 ; *CC(C*)(CC)COC(=O)c1ccccc1 ; *CCN(CC*)C(=O)C(C)(C)C]

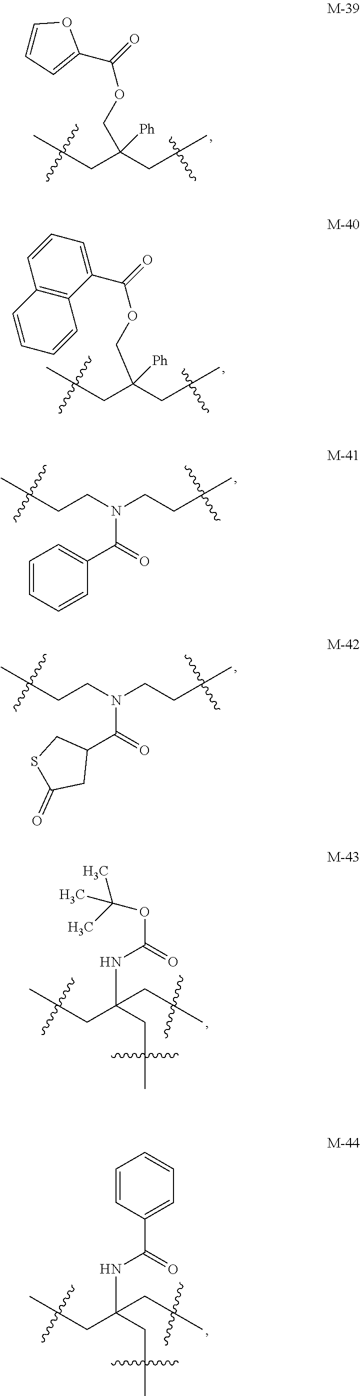 [SMILES: *CC(C*)(COC(=O)c1ccco1)c1ccccc1 ; *CC(C*)(COC(=O)c1cccc2ccccc12)c1ccccc1 ; *CCN(CC*)C(=O)c1ccccc1 ; *CCN(CC*)C(=O)C1CSC(=O)C1 ; *CC(C*)(C*)NC(=O)OC(C)(C)C ; *CC(C*)(C*)NC(=O)c1ccccc1]

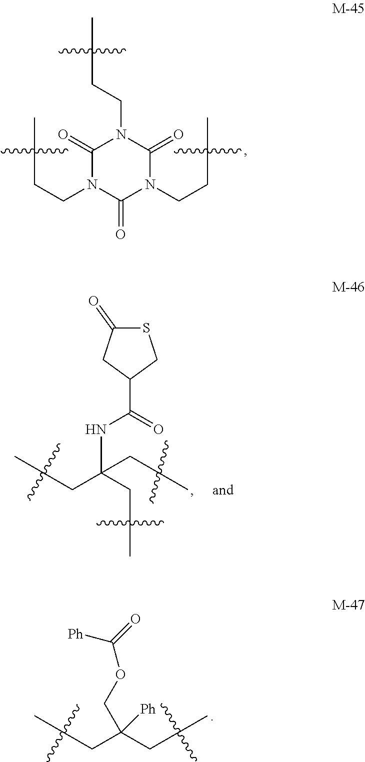 [SMILES: *CCn1c(=O)n(CC*)c(=O)n(CC*)c1=O ; *CC(C*)(C*)NC(=O)C1CSC(=O)C1 ; *CC(C*)(COC(=O)c1ccccc1)c1ccccc1]

3. The compound of claim 2 wherein
X is 2 or 3;
M is a di- or tri-valent core moiety;
R¹ is hydrogen or C₁-C₄ alkyl;
R² is hydrogen or C₁-C₄ alkyl;
R³ is hydrogen or C₁-C₄ alkyl;
R⁴ is hydrogen or C₁-C₄ alkyl;
R⁵ is hydrogen or C₁-C₄ alkyl;
R⁶ is hydrogen or methyl.

4. The compound of claim 3 wherein
X is 2;
M is a di-valent core moiety;
each G is independently O;
R¹ is hydrogen or methyl;
R² is hydrogen or methyl;
R³ is hydrogen or methyl;
R⁴ is hydrogen or methyl; and
R⁵ is hydrogen or methyl.

5. The compound of claim 4 wherein
M is di-valent core moiety comprising phenyl or pyridine, each phenyl or pyridine optionally substituted with up to 5 substituents independently selected from R^{A}; or a 5- to 6-membered fully unsaturated heterocyclic ring or an 6- to 10-membered heteroaromatic bicyclic ring system, each ring or ring system containing ring members selected from carbon atoms and 1 to 4 heteroatoms independently selected from up to 2 O, up to 2 S and up to 4 N atoms, wherein up to 3 carbon ring members are independently selected from C(═O) and C(═S), each ring or ring system optionally substituted with up to 5 substituents independently selected from R^{B} on carbon atom ring members and selected from R^{C} on nitrogen atom ring members; and the two attachment points to G are at any position on the phenyl, pyridine or R^{A}, 5- to 6-membered fully unsaturated heterocyclic ring or 6 to 10-membered heteroaromatic bicyclic ring system or R^{B} or R^{C};
each R^{A}, R^{B} and R^{C} are independendly selected from methyl, ethyl or cyclopropyl;
R¹ is hydrogen;
R² is hydrogen;
R³ is hydrogen;
R⁴ is hydrogen; and
R⁵ is hydrogen.

6. The compound of claim 5 wherein
M is selected from M-1, M-2, M-3, M-4, M-5, M-7, M-8, M-9, M-10, M-11, M-12, M-13, M-14, M-15, M-16, M-18, M-19, M-20, M-21, M-24 and M-27; and
each R^{A}, R^{B} and R^{C} are independendly selected from methyl.

7. The compound of claim 5 wherein
M is selected from the group consisting of M-6, M-17, M-22, M-23 and M-25;
R¹ is hydrogen;
R² is hydrogen;
R³ is hydrogen;
R⁴ is hydrogen; and
R⁵ is hydrogen.

8. The compound of claim 1 selected from the group consisting of
X is 2;
M is selected from the group consisting of M-2 or M-13;
each G is independently O;
R¹ is hydrogen;
R² is hydrogen;
R³ is hydrogen;
R⁴ is hydrogen; and
R⁵ is hydrogen.

9. The compound of claim 1 selected from the group consisting of
X is 3;
M is M-23;
each G is independently O;
R¹ is hydrogen;
R² is hydrogen;
R³ is hydrogen;
R⁴ is hydrogen; and
R⁵ is hydrogen.

10. A polymer microcapsule prepared from the compound of Formula 1 as defined in claim 1 and an amine selected from a di-amine or a tri-amine.

11. The polymer microcapsule prepared according to claim 10 wherein the amine is selected from a di-amine.

12. The polymer microcapsule prepared according to claim 11 wherein the di-amine is 1,6-hexamethylenediamine (HMDA).

13. The polymer microcapsule prepared according to claim 10 wherein the process further includes an acrylate.

14. The polymer microcapsule prepared according to claim 13 wherein the acrylate is trimethylolpropane triacrylate (TMPTA).

15. The polymer microcapsule of claim 10 wherein the polymer is prepared by the process comprising:
a) forming a mixture comprising:
at least one thiolactone pre-polymer compound of Formula 1; and
at least one di-amine or ti-amine compound;
b) reacting the at least one thiolactone pre-polymer compound of Formula 1 and the at least one di-amine or ti-amine compound to form the polymer reaction product of the at least one thiolactone pre-polymer compound of Formula 1 and the at least one di-amine or tri-amine compound in the presence of an agrochemical compound; and
c) microencapsulating the agrochemical compound with the reaction product.

16. The process of preparing the pre-polymer microcapsule of claim 15 further comprising wherein step b) further includes a cross-linker.
